(19) United States
(12) Patent Application Publication
(10) Pub. No.: US 2007/0190538 A1
(43) Pub. Date: Aug. 16, 2007

(54) Chimeric polymerases

(75) Inventors: Patrick K. Martin, Redwood City, CA (US); David A. Simpson, Redwood City, CA (US)

Correspondence Address: DECHERT LLP, P.O. BOX 10004, PALO ALTO, CA, 94303, US

(73) Assignee: Applera Corporation, Foster City, CA (US)

(21) Appl. No.: 11/496,596

(22) Filed: Jul. 31, 2006

Related U.S. Application Data
(60) Provisional application No. 60/704,013, filed on Jul. 29, 2005.

(51) Int. Cl. C12Q 1/68; C07H 21/04; C12P 21/06; C12N 9/22; C12N 15/74; C12N 1/21

(52) U.S. Cl. 435/6; 435/69.1; 435/199; 435/252.3; 435/471; 536/23.2

(57) ABSTRACT

Disclosed herein are chimeric polymerases and methods of making and using same.

### 1. CROSS REFERENCE TO RELATED APPLICATIONS

This application claims benefit under 35 U.S.C. § 119(e) to application Ser. No. 60/704,013, filed Jul. 29, 2005, the contents of which are incorporated herein by reference.

### 2. BACKGROUND

DNA polymerases with 3′→5′ exonuclease (proofreading) activity are the enzyme of choice for DNA amplification reactions where a high degree of fidelity is desired. The appeal of these polymerases is offset by their “read-ahead” activity which reduces processivity thereby reducing the yield of DNA amplification products. Read-ahead activity detects base-analogs that can be present in a DNA template and causes the polymerase to stall. Base-analogs arise in DNA as a result of various processes. For example, under thermocycling conditions, cytosine in DNA and dCTP monomers in solution deaminate and are thereby converted to uracil. Thus, uracil-containing DNA can arise from deamination of cytosine residues in a DNA template or by deamination of dCTP to dUTP and polymerase incorporation of the dUTP monomers into DNA. (Slupphaug et al. Anal Biochem. 1993; 211:164-169). Upon encountering uracil in a DNA template, the read-ahead activity causes the polymerase to stall upstream of the uracil residue. (Lasken et al. J Biol Chem. 1996; 271:17692-17696). Therefore, as the amount of uracil in DNA increases, the yield of amplification product decreases. Thus, there is a need in the art for DNA polymerases with reduced sensitivity to nucleotide analogs, such as uracil, that inhibit polymerase activity.

### 3. SUMMARY

These and other features of the present teachings are set forth herein.

The present disclosure provides chimeric polypeptides comprising heterologous amino acid sequences or domains. In some embodiments, a chimeric polypeptide can comprise a first domain having polymerizing activity joined to a second domain that reduces the sensitivity of the polymerizing domain to uracil. Therefore, disclosed herein are chimeric polymerases with reduced susceptibility to uracil poisoning. In various exemplary embodiments, the chimeric polymerases disclosed herein have reduced rates of dUTP incorporation into DNA and/or have reduced sensitivity to uracil in a DNA template. In various exemplary embodiments, a chimeric polymerase having one or more of these properties can comprise a polymerizing domain fused to an amino acid sequence having dUTPase activity and/or an amino acid sequence having double-stranded DNA binding activity.

In various exemplary embodiments, a domain having polymerizing activity can be a type A-, B-, C-, X-, or Y- family polymerase or a homolog or subsequence thereof suitable for catalyzing DNA polymerization in a template directed manner. In some embodiments, a domain having polymerizing activity can be a thermostable polymerase, such as, an Archaeal B-family DNA polymerase or an enzymatically active subsequence thereof. Non-limiting examples of Archaeal B-family DNA polymerases can include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like. Examples of Archaeal B-family DNA polymerases include, but are not limited to, Vent™, Deep Vent™, Pfu, KOD, Pfx, Therminator, and Tgo polymerases.

In various exemplary embodiments, a domain having dUTPase activity can be a full-length dUTPase or a homolog or subsequence thereof sufficient to catalyze the hydrolysis of dUTP to dUMP and pyrophosphate. A dUTPase can be of prokaryotic, eukaryotic, (including nuclear and mitochondrial isoforms), or viral origin. In some embodiments, a dUTPase can be thermostable. Therefore, in some embodiments, a dUTPase can be from various Archaea genera, as described herein or known in the art.

In some embodiments, a domain having double-stranded DNA binding activity can be any amino acid sequence that binds double-stranded DNA in a sequence independent manner. In some embodiments, a double-stranded DNA binding domain increases the processivity of a chimeric polymerase in a template. In some embodiments, an amino acid sequence comprising sequence-independent, double-stranded DNA binding activity can be thermostable, such as, an Archaeal sequence-independent, double-stranded DNA binding protein (dsDBP). Non-limiting examples of Archaeal dsDBPs include, Ape3192, Pae3192, Sso7d, Smj12, Alba-1 (e.g., Sso-10b-1, Sac10a), Alba-2, proliferating cell nuclear antigen (PCNA), including homologs and subsequences thereof.

In some embodiments, one or more mutations can be introduced into the sequence of a chimeric polypeptide to modify one or more activities of the various domains. Mutations can be any one or more of a substitution, insertion, and/or deletion of one or a plurality of amino acids. In various exemplary embodiments, a mutation can decrease the base analog detection or the 3′→5′ exonuclease activity of chimeric polymerases. In some embodiments, a mutation can be suitable to increase the types of non-natural nucleotide base analogs that can be incorporated into a DNA strand by a chimeric polymerase. In some embodiments, a mutation can modify the specific activity of a polymerizing domain of a chimeric polypeptide.

The chimeric polypeptides disclosed herein can be synthesized by various methods. In some embodiments, a chimeric polypeptide can be expressed by a host cell from a recombinant polynucleotide vector comprising a sequence that encodes for the chimeric polypeptide. The recombinant vector can be made by ligating the appropriate polynucleotide sequences encoding the various domains and operatively linking the encoding sequence to a constitutive or inducible promoter, as known in the art. In various exemplary embodiments, a cell suitable for expressing a chimeric polypeptide can be a prokaryotic or eukaryotic cell. In some embodiments the domains comprising a chimeric polypeptide can be joined by chemical conjugation using one or more hetero-bifinctional coupling reagents, which can be cleavable or non-cleavable. Other non-limiting examples of coupling methods can utilize intermolecular disulfide bonds or thioether linkages. In some embodiments, the domains of a chimeric polypeptide can be joined by non-covalent interactions, such as, ionic interactions. (see, e.g. U.S. Pat. No. 6,627,424, WO/2001/92501).

The chimeric polypeptides disclosed herein find use in various methods, such as, synthesizing, analyzing, sequencing, modifying, and amplifying polynucleotide sequences. In some embodiments, a method of synthesizing a polynucleotide can comprise contacting a polynucleotide template with a primer and a chimeric polypeptide under conditions suitable for the chimeric polypeptide to extend the primer in a template directed manner. In some embodiments, a method of amplifying a target polynucleotide sequence comprises contacting a target sequence with a primer and a chimeric polypeptide under thermocycling conditions suitable for the chimeric polypeptide to amplify the target sequence. In some embodiments, a method of sequencing a polynucleotide can comprise contacting a target sequence with a primer and a chimeric polypeptide in the presence of nucleotide triphosphates and one or more chain terminating agents to generate chain terminated fragments; and determining the sequence of the polynucleotide by analyzing the fragments.

### 4. BRIEF DESCRIPTION OF THE DRAWINGS

The skilled artisan will understand that the drawings, described below, are for illustration purposes only and are not intended to limit the scope of the present disclosure in any way.

FIG. 1 shows an alignment of the amino acid sequences of a region of the read-ahead domain of Archaeal B-family polymerases. (Connolly et al. Biochem Soc Trans. 2003; 31:699; Fogg et al. Nature Struct Biol. 2002; 9:922-927; Shuttleworth et al. J Mol Biol. 2004; 337:621-634). The numbering of amino acids, such as, the amino acid residues at positions V93 and P115 including residues corresponding thereto is based on the number of amino acids of the full-length, mature polymerase B of Pyrococcus furiosus (P_fur, GenBank BAA02362, D12983 (SEQ ID NO:2). (Pyrococcus abyssi (P_abyssi (SEQ ID NO:1), GenBank P77916, AL096836); Pyrococcus species GB-D (P_GBD (SEQ ID NO:3), DEEP VENT™, GenBank PSU00707, AAA67131); Pyrococcus glycovorans (P_glycov (SEQ ID NO:4), GenBank AJ250335, CAC12849, TGL250335); Pyrococcus spp. ST700 (P_ST700 (SEQ ID NO:5), GenBank AJ250332, CAC12847); Thermococcus 9-degrees-Nm (T_{—}9oNm (SEQ ID NO:6), Thermococcus sp. 9°N-7, GenBank U47108, AAA88769, TSU47108, **Q56366); Thermococcus fumicolans (T_fum (SEQ ID NO:7), GenBank TFDPOLEND, CAA93738); Thermococcus gorgonarius (T_gorg (SEQ ID NO:8), GenBank P56689); Thermococcus hydrothermalis (T_hydro (SEQ ID NO:9), GenBank THY245819, CAC18555); Thermococcus spp. JDF-3 (T_JDF3 (SEQ ID NO:10), GenBank AX135456; WO0132887); Thermococcus kodakarensis (T_KOD (SEQ ID NO:11), GenBank BAA06142, BD175553); Thermococcus litoralis (T_lit (SEQ ID NO:12), VENT™, GenBank AAA72101); Thermococcus profundus (T_profundus (SEQ ID NO:13), GenBank E14137; CAPLUS/REGISTRY Database 199455-28-2 (T. profundus strain DT5432 (9CI)); JP1997275985A)).

FIG. 2 Panel A provides a cartoon of a non-limiting example of an Archaeal type-B DNA polymerase comprising a polymerizing domain and a 3′→5′ exonuclease domain (3′→5′ exo). Panels B-E provide cartoons of non-limiting examples of chimeric polymerases comprising Archael type-B DNA polymerizing domain jointed to a dUTPase and/or a non-specific dsDNA binding domain (“BP”) and/or a 3′→5′ exo domains.

FIG. 3 shows the amino acid sequences of non-specific DNA binding protein Sso7d which is present in the Sulfolobus sulfataricus P2 genome (see GenBank NC 002754) in three nearly-identical open reading frames: Sso10610 (SEQ ID NO:14), Sso9180 (SEQ ID NO:15), Sso9535 (SEQ ID NO:16). (Gao et al. Nature Struct Biol. 1998; 5:782-786).

FIG. 4 shows the amino acid sequence of non-specific DNA binding protein Smj12 of the Sulfolobus sulfataricus P2 genome (see GenBank NC 002754) open reading frame Sso0458 (SEQ ID NO:17). (Napoli et al. J Biol Chem. 2001; 276:10745-10752).

FIG. 5 shows the amino acid sequence of non-specific DNA binding protein Alba-1 (Sso-10b-1, Sac10a) of the Sulfolobus sulfataricus P2 genome (see GenBank NC_{—}002754) open reading frame Sso0962 (SEQ ID NO:18). (Wardleworth et al. EMBO J. 2002; 21:4654-4652).

FIG. 6 shows the amino acid sequence of non-specific DNA binding protein Alba-2 of the Sulfolobus sulfataricus P2 genome (see GenBank NC 002754) open reading frame Sso6877 (SEQ ID NO:19). (Chou et al. J Bacteriol. 2003; 185:4066-4073).

FIG. 7 shows the amino acid sequence of proliferating cell nuclear antigen homolog of P. furiosus (Pfu PCNA (SEQ ID NO:20)) (GenBank AB017486, BAA33020). (Cann et al. J Bacteriol. 1999; 181-6591-6599; Motz et al. J Biol Chem. 2002; 277:16179-16188).

FIG. 8 shows the amino acid sequence of non-specific DNA binding proteins Pae3192 (SEQ ID NO:21), Pae3289 (SEQ ID NO:22), and PaeO384 (SEQ ID NO:23) of Pyrobaculum aerophilum strain IM2 (GenBank NC_{—}003364).

FIG. 9 shows the amino acid sequence of non-specific DNA binding protein Ape3192 (SEQ ID NO:24) of Aeropyrum pernix (GenBank NC_{—}000854).

FIG. 10 shows the amino acid sequence of Pyrococcus furiosus DNA polymerase (SEQ ID NO:25) (Pfu, GenBank D12983, BAA02362)

FIG. 11 shows the nucleic acid sequence encoding the amino acid sequence of Thermococcus kodakarensis strain KOD1 DNA polymerase (SEQ ID NO:26) (GenBank BD175553).

FIG. 12 shows the amino acid sequence of VENT™ DNA polymerase (SEQ ID NO:27) (GenBank AAA72101).

FIG. 13 shows the amino acid sequence of DEEP VENT™ DNA polymerase (SEQ ID NO:28) (GenBank AAA67131).

FIG. 14 shows amino acid sequence of Tgo DNA polymerase (SEQ ID NO:29) (GenBank P56689, Hopfner et al. Proc Natl Acad Sci USA. 1999 Mar. 30; 96(7):3600-5).

FIG. 15 shows the amino acid sequence of Archaeoglobus fulgidus DNA polymerase (SEQ ID NO:30) (GenBank 029753).

FIG. 16 shows an alignment of the amino acid sequence of Archaeal DNA polymerases. The numbering of amino acids, such as, the amino acid residues at positions 247, 265, 408, and 485 is based on the number of amino acids of the full-length polymerase B of Pyrococcus furiosus (GenBank BAA02362); Pyrococcus abyssi (GenBank P77916); Pyrococcus furiosus (GenBank BAA02362); Pyrococcus species GB-D (GenBank PSU00707)); Pyrococcus glycovorans (GenBank CAC12849); Pyrococcus sp. ST700 (GenBank CAC12847); Thermococcus 9-degrees-Nm (Thermococcus sp. 9oN-7 (GenBank AAA887669); Thermococcus fumicolans (GenBank CAA93738); Thermococcus gorgonarius (GenBank P56689, 1QQCA, 1D5AA); Thermococcus hydrothermalis (GenBank CAC 18555); Thermococcus sp. JDF-3 (GenBank AX135456; WO0132887); Thermococcus kodakarensis (GenBank BAA06142); Thermococcus litoralis (GenBank AAA72101); Thermococcus profundus (GenBank E14137; JP1997275985A). Panel A shows Forked Point substitutions (P_abyssi (SEQ ID NO:46), P_fur (SEQ ID NO:47), P_GBD (SEQ ID NO:48), P_glycov (SEQ ID NO:49), P_ST700 (SEQ ID NO:50), T_{—}9oNm (SEQ ID NO:51), T_fum (SEQ ID NO:52), T_gorg (SEQ ID NO:53), T_hydro (SEQ ID NO:54), T_JDF3 (SEQ ID NO:55), T_KOD (SEQ ID NO:56), T_lit (SEQ ID NO:57), T_profundus (SEQ ID NO:58)). Panel B shows Finger substitutions (P_abyssi (SEQ ID NO:59), P_fur (SEQ ID NO:60), P_GBD (SEQ ID NO:61), P_glycov (SEQ ID NO:62), P_ST700 (SEQ ID NO:63), T_{—}9oNm (SEQ ID NO:64), T_fum (SEQ ID NO:65), T_gorg (SEQ ID NO:66), T_hydro (SEQ ID NO:67), T_JDF3 (SEQ ID NO:68), T_KOD (SEQ ID NO:69), T_lit (SEQ ID NO:70), T_profundus (SEQ ID NO:71)). See FIG. 2 for key.

FIG. 17 shows the results of a PCR reaction performed in the presence of varying dTTP/dUTP ratios using a non-limiting example of a chimeric polymerase comprising: (i) Pfu polymerizing domain fused at its carboxy terminus to non-specific DNA binding protein Pae3192; and (ii) a chimeric polymerase comprising Pfu polymerizing domain fused at its carboxy terminus with non-specific DNA binding protein Pae3192 and further comprising substitution of a glutamine (Q) for valine-93 (V93Q, see FIG. 1), which substantially inactivates the base analog detection domain.

FIG. 18 shows oligonucleotides utilized in the assembly of a polynucleotide that encodes a thermostable dUTPase. (dut1 (SEQ ID NO:31), dut2 (SEQ ID NO:32), dut3 (SEQ ID NO:33), dut4 (SEQ ID NO:34), dut5 (SEQ ID NO:35), dut6 (SEQ ID NO:36), dut7 (SEQ ID NO:37), dut8 (SEQ ID NO:38), duta (SEQ ID NO:39), dutb (SEQ ID NO:40), dutc (SEQ ID NO:41), dutd (SEQ ID NO:42), dute (SEQ ID NO:43), dutf (SEQ ID NO:44), dutg (SEQ ID NO:45)).

FIG. 19 shows the DNA sequence encoding chimeric polymerase comprising an amino terminal histidine tail: His₁₀-Pfu-Ape3192(V93Q) (SEQ ID NO:72).

FIG. 20 shows the amino acid sequence of chimeric polymerase comprising an amino terminal histidine tail: His₁₀-Pfu-Ape3192(V93Q) (SEQ ID NO:73).

FIG. 21 shows the amino acid sequence of chimeric polymerase comprising an amino terminal histidine tail: His₁₀-Pfu-Pae3192(V93Q) (SEQ ID NO:74).

FIG. 22 shows the DNA sequence encoding chimeric polymerase comprising an amino terminal histidine tail: His₁₀-Pfu-Pae3192(V93Q) (SEQ ID NO:75).

### 5. DETAILED DESCRIPTION

It is to be understood that both the foregoing general description, including the drawings, and the following detailed description are exemplary and explanatory only and are not restrictive of this disclosure. In this disclosure, the use of the singular includes the plural unless specifically stated otherwise. Also, the use of “or” means “and/or” unless stated otherwise. Similarly, “comprise,” “comprises,” “comprising” “include,” “includes,” and “including” are not intended to be limiting. Terms such as “element” or “component” encompass both elements and components comprising one unit and elements or components that comprise more than one unit unless specifically stated others. The sectional heads used herein are for organizational purposes only and are not to be construed as limiting the subject matter described. All references and portions of references cited, including but not limited to patents, patent applications, articles, books, and treatises are hereby expressly incorporated by reference in their entirely for any purpose. In the event that one or more of the incorporated references contradicts this disclosure, this disclosure controls.

5.2 Definitions

“Protein,” “polypeptide,” “oligopeptide,” and “peptide” are used interchangeably to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

“Nucleobase polymer” and “oligomer” refer to two or more nucleobases connected by linkages that permit the resultant nucleobase polymer or oligomer to hybridize to a polynucleotide having a complementary nucleobase sequence. Nucleobase polymers or oligomers include, but are not limited to, poly- and oligonucleotides (e.g., DNA and RNA polymers and oligomers), poly- and oligonucleotide analogs and poly- and oligonucleotide mimics, such as polyamide or peptide nucleic acids. Nucleobase polymer and oligomer include, but are not limited to, mixed poly- and oligonucleotides (e.g., a combination of DNA, RNA, and/or peptide nucleic acids and the like). Nucleobase polymers or oligomers can vary in size from a few nucleobases, from about 2 to about 40 nucleobases, to about several hundred nucleobases, to about several thousand nucleobases, or more.

“Polynucleotide” and “oligonucleotide” refer to nucleobase polymers or oligomers in which the nucleobases are connected by sugar phosphate linkages (e.g., a sugar-phosphate backbone). Exemplary poly- and oligonucleotides include polymers of 2′-deoxyribonucleotides (e.g., DNA) and polymers of ribonucleotides (e.g., RNA). In various exemplary embodiments, a polynucleotide may be composed entirely of ribonucleotides, entirely of 2′-deoxyribonucleotides, or combinations thereof.

“Polynucleotide analog” and “oligonucleotide analog” refer to nucleobase polymers or oligomers in which the nucleobases are connected by a sugar phosphate backbone comprising one or more sugar phosphate analogs. Typical sugar phosphate analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2′-deoxyribose or ribose, nucleobase polymers having positively charged sugar-guanidyl interlinkages such as those described in U.S. Pat. Nos. 6,013,785, 5,696,253 (see also, Dagani, 1995, Chem. & Eng. News 4-5:1153; Dempey et al., 1995, J. Am. Chem. Soc. 117:6140-6141). Such positively charged analogues in which the sugar is 2′ deoxyribose are referred to as “DNGs,” whereas those in which the sugar is ribose are referred to as “RNGs.” Specifically included within the definition of poly- and oligonucleotide analogs are locked nucleic acids (LNAs; see, e.g., Elayadi et al. 2002, Biochemistry 41:9973-9981; Koshkin et al., 1998, J. Am. Chem. Soc. 120:13252-3; Koshkin et al., 1998, Tetrahedron Letters, 39:4381-4384; Jumar et al., 1998, Bioorganic & Medicinal Chemistry Letters 8:2219-2222; Singh and Wengel, 1998, Chem. Commun., 12:1247-1248; WO 00/56746; WO 02/28875; and WO 01/48190.

“Polynucleotide mimic” and “oligonucleotide mimic” refers to a nucleobase polymer or oligomer in which one or more of the backbone sugar-phosphate linkages is replaced with a sugar-phosphate analog. Such mimics are capable of hybridizing to complementary polynucleotides or oligonucleotides, or polynucleotide or oligonucleotide analogs or to other polynucleotide or oligonucleotide mimics, and may include backbones comprising one or more of the following linkages: positively charged polyamide backbone with alkylamine side chains as described in U.S. Pat. Nos. 5,786,461, 5,766,855, 5,719,262, 5,539,082 and WO 98/03542 (see also, Haaima et al., 1996, Angewandte Chemie Int'l Ed. in English 35:1939-1942; Lesnick et al., 1997, Nucleotid. 16:1775-1779; D'Costa et al., 1999, Org. Lett. 1:1513-1516; Nielsen, 1999, Curr. Opin. Biotechnol. 10:71-75); uncharged polyamide backbones as described in WO92/20702 and U.S. Pat. No. 5,539,082; uncharged morpholino-phosphoramidate backbones as described in U.S. Pat. Nos. 5,698,685, 5,470,974, 5,378,841, and 5,185,144 (see also, Wages et al., 1997, BioTechniques 23:1116-1121); peptide-based nucleic acid mimic backbones (see, e.g., U.S. Pat. No. 5,698,685); carbamate backbones (see, e.g,, Stirchak and Summerton, 1987, J. Org. Chem. 52:4202); amide backbones (see, e.g., Lebreton, 1994, Synlett. February, 1994:137); methylhydroxyl amine backbones (see, e.g., Vasseur et al., 1992, J. Am. Chem. Soc. 114:4006); 3′-thioformacetal backbones (see, e.g., Jones et al., 1993, J. Org. Chem. 58:2983) and sulfamate backbones (see, e.g., U.S. Pat. No. 5,470,967). All of the preceding references are herein incorporated by reference.

“Fused,” “joined” and grammatical equivalents are used herein refers to linkage of heterologous amino acid or polynucleotide sequences. Thus, “fused” refers to any method known in the art for functionally connecting polypeptide and/or polynucleotide sequences, such as, domains, including but not limited to recombinant fusion with or without intervening linking sequence(s), domain(s) and the like, non-covalent association, and covalent bonding.

“Chimeric polypeptide” and grammatical equivalents refers to a polypeptide comprising two or more heterologous domains, amino acid sequences, peptides, and/or proteins joined either covalently or non-covalently to produce a polypeptide that does not occur in nature. Therfore, a chimera includes a fusion of a first amino acid sequence joined to a second amino acid sequence, wherein the first and second amino acid sequences are not found in the same relationship in nature. As used herein, “joined” and “fused” refer to any method known in the art for functionally connecting polypeptide domains, including without limitation recombinant fusion with or without intervening domain(s), sequence(s) and the like, intein-mediated fusion, non-covalent association, and covalent bonding, including disulfide bonding, hydrogen bonding, electrostatic bonding, and conformational bonding.

“Heterologous” as used herein with reference to chimeric polypeptides refers to two or more domains or sequences that are not found in the same relationship to each other in nature. Therefore, a fusion of two or more heterologous domains or sequences from unrelated proteins can yield a chimeric polypeptide.

“Domain” as used herein refers to an amino acid sequence of a chimeric polypeptide comprising one or more defined finctions or properties.

“Nucleic acid polymerase” or “polymerase” refers to a polypeptide that catalyzes the synthesis of a polynucleotide using an existing polynucleotide as a template. Therefore, in various exemplary embodiments, a polymerase can be a DNA-dependent DNA polymerase, an RNA-dependent DNA polymerase, an RNA-dependent RNA polymerase, etc.

“DNA polymerase” as used herein refers to a nucleic acid polymerase capable of catalyzing the synthesis of DNA using a polynucleotide template.

“Thermostable” as used herein refers to a polypeptide which does not become irreversibly denatured (inactivated) when subjected to elevated temperatures for the time necessary to effect denaturation of double-stranded nucleic acids. The heating conditions necessary for nucleic acid denaturation are well known in the art and are exemplified in U.S. Pat. Nos. 4,683,202 and 4,683,195. Irreversible denaturation for purposes herein refers to permanent and at least substantial loss of activity, structure, or function. In various exemplary embodiments, a thermostable polypeptide is not irreversibly denatured following incubation of at least about 50° C., 60° C., 70° C., 80° C., or 90° C., or higher for 3, 4, 5, 6, 7, 8, 9, 10, or more minutes.

“Polymerase activity” refers to the activity of a nucleic acid polymerase in catalyzing the template-directed synthesis of a polynucleotide. Polymerase activity can be measured using various techniques and methods known in the art. For example, serial dilutions of polymerase can be prepared in dilution buffer (20 mM Tris.Cl, pH 8.0, 50 mM KCl, 0.5% NP 40, and 0.5% Tween-20). For each dilution, 5 μl can be removed and added to 45 μl of a reaction mixture containing 25 mM TAPS (pH 9.25), 50 mM KCl, 2 mM MgCl₂, 0.2 mM dATP, 0.2 mM dGTP, 0.2 mM dTTP, 0.1 mM dCTP, 12.5 μg activated DNA, 100 μM [α-³²P]dCTP (0.05 μCi/nmol) and sterile deionized water. The reaction mixtures can be incubated at 37° C. (or 74° C. for thermostable DNA polymerases) for 10 minutes and then stopped by immediately cooling the reaction to 4° C. and adding 10 μl of ice-cold 60 mM EDTA. A 25 μl aliquot can be removed from each reaction mixture. Unincorporated radioactively labeled dCTP can be removed from each aliquot by gel filtration (Centri-Sep, Princeton Separations, Adelphia, N.J.). The column eluate can be mixed with scintillation fluid (1 ml). Radioactivity in the column eluate is quantified with a scintillation counter to determine the amount of product synthesized by the polymerase. One unit of polymerase activity can be defined as the amount of polymerase necessary to synthesize 10 nmole of product in 30 minutes. (Lawyer et al. (1989) J. Biol. Chem. 264:6427-647). Other methods of measuring polymerase activity are known in the art (see, e.g. Sambrook et al. (2001) Molecular Cloning: A Laboratory Manual (3^{rd} ed., Cold Spring Harbor Laboratory Press, NY)).

“Processivity” refers to the ability of a polymerase to perform a sequence of polymerization steps without intervening dissociation of the polymerase from the growing polynucleotide strand. Thus, processivity can be measured by the number of nucleotides a polymerase can add to a primer terminus during a polymerization cycle. “Polymerization cycle” includes the steps of “diffusion of the enzyme to the primer terminus . . . the ordered binding of a nucleotide, base pairing with template, covalent linkage to the primer terminus, and then translocation of the enzyme to the newly created primer terminus. The enzyme either dissociates at this point to complete the cycle or continues processively.” (Kornberg, DNA Replication, p. 122 (Freeman & Co. 1980 (ISBN: 0716711028)). Therefore, processivity refers to the number of nucleotides added by a polymerase to an oligonucleotide primer while the polymerase is in contact with the primer and template during a polymerization cycle.

“Nucleic acid binding activity” refers to the activity of a polypeptide in binding nucleic acid in a two band-shift assay. For example, in some embodiments (based on the assay of Guagliardi et al. (1997) J. Mol. Biol. 267:841-848), double-stranded nucleic acid (the 452-bp HindIII-EcoRV fragment from the S. solfataricus lacS gene) is labeled with ³²P to a specific activity of at least about 2.5×10⁷ cpm/ug (or at least about 4000 cpm/fmol) using standard methods. See, e.g., Sambrook et al. (2001) Molecular Cloning: A Laboratory Manual (₃^{rd} ed., Cold Spring Harbor Laboratory Press, NY) at 9.63-9.75 (describing end-labeling of nucleic acids). A reaction mixture is prepared containing at least about 0.5 μg of the polypeptide in about 10 μl of binding buffer (50 mM sodium phosphate buffer (pH 8.0), 10% glycerol, 25 mM KCl, 25 mM MgCl₂). The reaction mixture is heated to 37° C. for 10 min. About 1×10⁴ to 5×10⁴ cpm (or about 0.5-2 ng) of the labeled double-stranded nucleic acid is added to the reaction mixture and incubated for an additional 10 min. The reaction mixture is loaded onto a native polyacrylamide gel in 0.5× Tris-borate buffer. The reaction mixture is subjected to electrophoresis at room temperature. The gel is dried and subjected to autoradiography using standard methods. Any detectable decrease in the mobility of the labeled double-stranded nucleic acid indicates formation of a binding complex between the polypeptide and the double-stranded nucleic acid. Such nucleic acid binding activity may be quantified using standard densitometric methods to measure the amount of radioactivity in the binding complex relative to the total amount of radioactivity in the initial reaction mixture.

In some embodiments, (based on the assay of Mai et al. (1998) J. Bacteriol. 180:2560-2563), about 0.5 μg each of negatively supercoiled circular pBluescript KS(−) plasmid and nicked circular pBluescript KS(−) plasmid (Stratagene, La Jolla, Calif.) are mixed with a polypeptide at a polypeptide/DNA mass ratio of about ≧2.6. The mixture is incubated for 10 min at 40° C. The mixture is subjected to 0.8% agarose gel electrophoresis. DNA is visualized using an appropriate dye. Any detectable decrease in the mobility of the negatively supercoiled circular plasmid and/or nicked circular plasmid indicates formation of a binding complex between the polypeptide and the plasmid.

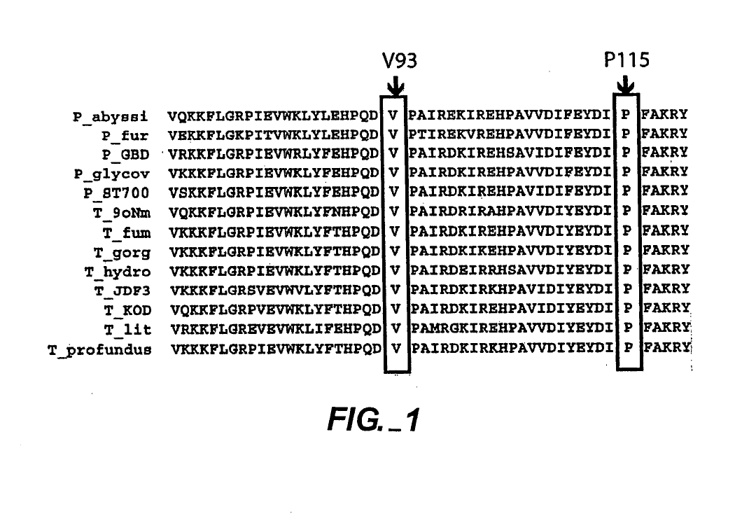

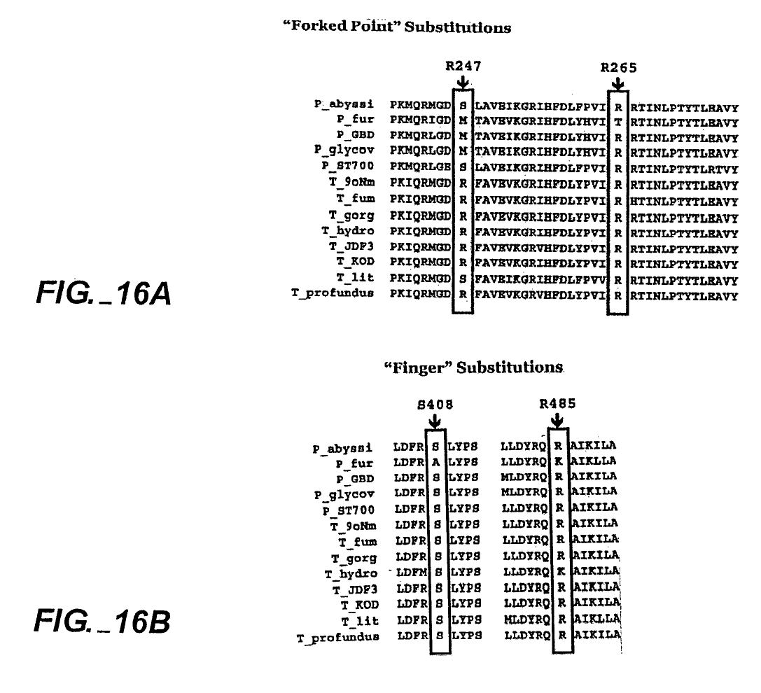

“Corresponding” as used herein refers to being similar or equivalent in character, structure, or function. Therefore, “corresponding amino acid” refers to an amino acid at a position in a polypeptide that is similar or equivalent in character, structure, or function to an amino acid in another polypeptide. In some embodiments, corresponding amino acids in two or more polypeptides can be identified by aligning polypeptide sequences using various algorithms as known in the art. (see, e.g. FIG. 1, FIG. 16A and 16B). In some embodiments, corresponding amino acids can be identified by aligning the polynucleotide sequences encoding the polypeptides. Algorithms suitable for aligning polypeptide or polynucleotide sequences in include the algorithms of Smith & Waterman, Adv. Appl. Math. 1981; 2:482, Needleman & Wunsch, J. Mol. Biol. 1970; 48:443, Pearson & Lipman, Proc Natl Acad Sci USA. 1998; 85:2444 and computerized implementations of these algorithms (e.g., GAP, BESTFIT, FASTA, and TFASTA). In some embodiments, sequence can be aligned by manually by visual inspection (see, e.g., Current Protocols in Molecular Biology (Ausubel et al., eds. 1995 supplement)). Other algorithms include PILEUP (Feng & Doolittle. J. Mol. Evol. 1987: 35:351-360; Devereaux et al., Nuc. Acids Res. 1984; 12:387-395), BLAST and BLAST 2.0 algorithms, which are described in Altschul et al. Nuc. Acids Res. 1977; 25:3389-3402; Altschul et al. J Mol Biol. 1990; 215:403-410; and; Karlin & Altschul. Proc. Natl. Acad. Sci. USA 1993; 90:5873-5787. Software for performing BLAST analyses is publicly available through the National Center for Biotechnology Information. In various exemplary embodiments, the default parameters of each of the alignment algorithms can be used.

Similarly, “corresponding nucleotides” can be identified by aligning two or more polynucleotide sequences using, for example, the Basic Local Alignment Search Tool (BLAST) engine. (Tatusova et al. (1999) FEMS Microbiol Lett. 174:247-250). The BLAST engine (version 2.2.10) is available to the public at the National Center for Biotechnology Information (NCBI), Bethesda, Md. To align two polynucleotide sequences, the “Blast 2 Sequences” tool can be used, which employs the “blastn” program with parameters set at default values (Matrix: not applicable; Reward for match: 1; Penalty for mismatch: −2; Open gap: 5 penalties; Extension gap: 2 penalties; Gap_x dropoff: 50; Expect: 10.0; Word size: 11; Filter: On).

“Native sequence” as used herein refers to a polynucleotide or amino acid isolated from a naturally occurring source. Included within “native sequence” are recombinant forms of a native polypeptide or polynucleotide which have a sequence identical to the native form.

“Mutant” or “variant” as used herein refers to an amino acid or polynucleotide sequence which has been altered by substitution, insertion, deletion and/or chemical modification. In some embodiments, a mutant or variant sequence can have increased, decreased, or substantially similar activities or properties in comparison to the parental sequence. In various exemplary embodiments, a “parental sequence” can be a wild-type sequence or another mutant or variant sequence. Exemplary activities or properties include but are not limited to polymerization, 3′→5′ exonuclease activity, base analog detection activities, such as uracil detection in DNA and inosine detection. A “mutant” or “variant” polymerase can be a chimeric polypeptide, such as a chimeric polymerase, as described herein.

“Host cell” as used herein refers to both single-cell prokaryote and eukaryote organisms such as bacteria, yeast, archaea, actinomycetes and single cells from higher order plants or animals grown in cell culture.

“Expression vector” as used herein refers to polynucleotide sequences containing a desired polypeptide coding sequence and control sequences in operable linkage, so that host cells transformed with polynucleotide sequences are capable of producing the encoded proteins either constitutively or via induction.

“Primer” as used herein refers to an oligonucleotide, whether natural or synthetic, which is capable of hybridizing to a template in a manner suitable to form a substrate for a polymerase. The appropriate length of a primer can vary by generally from about 15 to about 35 nucleotides. A primer need not reflect the exact sequence of the template but must be sufficiently complementary to hybridize with a template under polymerization conditions. In some embodiments, a primer can comprise a label suitable for detection by spectroscopic, photochemical, biochemical, immunochemical, or chemical methods.

“Archaeal” DNA polymerase refers to DNA polymerases that belong to either the Family B/pol I-type group (e.g., Pfu, KOD, Pfx, Vent, Deep Vent, Tgo, Pwo) or the pol II group (e.g., Pyrococcus furiosus DP1/DP2 2-subunit DNA polymerase). In some embodiments, “Archaeal” DNA polymerases can be thermostable Archaeal DNA polymerases and include, but are not limited to, DNA polymerases isolated from Pyrococcus species (e.g. , furiosus, species GB-D, woesii, abysii, horikoshii), Thermococcus species (kodakaraensis KODI, litoralis, species 9 degrees North-7, species JDF-3, gorgonarius), Pyrodictium occultum, and Archaeoglobus fulgidus. Archaeal pol I DNA polymerase group can be commercially available, including Pfu (Stratagene), KOD (Toyobo), Pfx (Life Technologies, Inc.), Vent (New England BioLabs), Deep Vent (New England BioLabs), Tgo (Roche), and Pwo (Roche). Additional archaea related to those listed above are described in the following references: Archaea: A Laboratory Manual (Robb, F. T. and Place, A. R., eds.), Cold Spring Harbor Laboratory Press, Cold Spring Harbor, N.Y., 1995.

5.3 Exemplary Embodiments

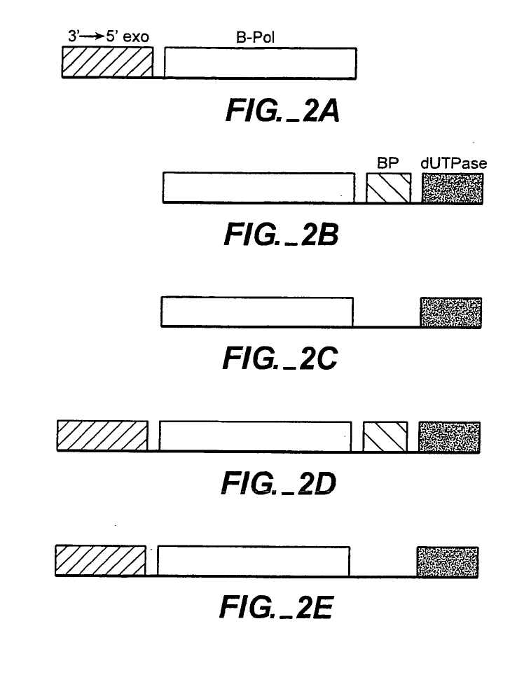

The present disclosure provides chimeric polypeptides comprising fusions of a DNA polymerizing domain and a heterologous domain to produce chimeric polymerases with reduced sensitivity to uracil. In some embodiments, a polymerizing domain can be fused to a dUTPase domain which converts dUTP to dUMP and pyrophosphate. dUMP and pyrophosphate are not suitable substrates for DNA polymerization and, therefore, are not utilized by the polymerizing domain. Accordingly, in some embodiments a chimeric polymerase can reduce the concentration of dUTP in a polymerization reaction before it can be incorporated into a newly synthesized DNA strand. As a result, the frequency or probability of polymerase stalling upon contacting a uracil-containing DNA can be substantially reduced. In some embodiments, chimeric polymerases with reduced sensitivity to uracil-containing DNA can comprise a fusion of a polymerizing domain and a heterologous domain that increases polymerase processivity (i.e., a processivity domain). Therefore, in some embodiments, a chimeric polymerase can substantially elide uracil-containing DNA. In some embodiments, a chimeric polymerase can comprise polymerizing, dUTPase, and processivity domains. In some embodiments, a chimeric polymerase can comprising one or more mutations to further decrease sensitivity to uracil and/or other types of base analogs that can be present in DNA templates. (FIG. 2A-E, 19-22).

Thus, “chimeric polymerase” as used herein refers to a polypeptide that does not occur in nature that comprises a fusion of two or more heterologous amino acid sequences or domains. Therefore, excluded from the definition of chimeric polymerases are naturally-occurring polypeptide fusions. These naturally-occurring fusions can be produced by various mechanisms, as known by the skilled artisan. For example, naturally-occurring fusions can be encoded by the genomes of various organisms, such as, viruses. Generally, naturally-occurring fusions can be post-translationally processed, for example, by viral and/or cellular proteases to yield discrete proteins. Non-limiting examples of naturally-occurring fusions are produced by retroviruses (e.g., pol, gag-pol, gag-pro, gag-pro-pol), togaviruses (e.g., nsP1-nsP2-nsp3-nsP4), picomaviruses (e.g., P1-P2-P3), and flaviviruses (e.g., C-prM-E-NS1-NS2A-NS3-NS4A-NS4B-NS5) etc. (Bannert. Proc Natl Acad Sci USA. 2004; 101:14572; Fields Virology 685-840, 895-1162, 1871-2140 (Knipe & Howley, editors-in-chief, 4^{th} ed., Lippincott Williams & Wilkins 2001 (ISBN: 0781718325); McGeoch. Nucl Acids Res. 1990; 18:4105-4110).

In contrast, the chimeric polymerases disclosed herein are hybrids that are engineered to contain elements or properties of two or more heterologous, donor polypeptides. The donor polypeptides can be from the same or different organisms (e.g., strains, subspecies, species, genera, families, kingdoms, etc.), can have distinct or related properties, can comprise native or mutant sequences, and can comprise the full-length polypeptide or one or more subsequences or fragments or domains thereof. The number and type of amino acid sequences from donor polypeptides that can be fused can be selected at the discretion of the practitioner.

“Polymerizing domain” as used herein refers to an amino acid sequence capable of catalyzing the synthesis of a polynucleotide using an existing polynucleotide strand as a template. Therefore, in various exemplary embodiments, a polymerizing domain can be a full-length polymerase or any fragment thereof capable of catalyzing polynucleotide synthesis in a template directed manner with or without the use of auxiliary proteins as known in the art (see, e.g. Komberg, DNA Replication (ISBN: 0716720035); Friedberg et al. DNA Repair And Mutagenesis (ISBN: 1555813194); Alberts et al. Molecular. Biology of the Cell, Fourth Edition (ISBN: 0815332181)). As the skilled artisan will appreciate, substrates suitable for polymerization include an oligonucleotide primer annealed to a template in a manner suitable for the template to form a 5′ overhang relative to the 3′ terminus of the primer (i.e., a primed template strand). Under suitable conditions as known in the art, a polymerizing domain utilizes nucleotide triphosphates to extend the 3′ terminus of the annealed primer. The sequence of the template directs the incorporation of nucleotides into the nascent strand to yield a polynucleotide that is the reverse complement of the template. Reaction conditions suitable for polymerization are well-known in the art and vary depending on the properties of the polymerizing domain, as described below. Other parameters include but are not limited to the composition of the nucleotide triphosphates (e.g., dNTPs, rNTPs), the template and primer (e.g., DNA, RNA), cofactors (e.g., divalent metal ions), ionic strength, pH, and temperature. (Innis et al. PCR Protocols: A Guide to Methods and Applications 1-482 (Academic Press (ISBN: 0123721814); Sambrook & Russell, Molecular Cloning: A Laboratory Manual 7.75-8.126, A4.11-A4.29 (3d Cold Spring Harbor Laboratory Press (ISBN: 0879695773)).

Polyrnerizing domains suitable for use as a chimeric polypeptide can be any of the various polymerases of eukaryotic and prokaryotic cells (e.g., archaebacteria, eubacteria), mitochondria, and viruses. In some embodiments, a polymerizing domain can be a DNA polymerizing domain of an A, B, C, D, X, Y or other polymerase family. The A, B, and C polymerase families are classified based on their amino acid sequence homology with the product of the polA, polB, or polC gene of E. coli that encode, respectively, for DNA polymerase I, II, and III (alpha subunit). The properties and enzymatic activities of each family of polymerase is known in the art. (Braithwaite et al. Nucleic Acids Res. 1993 Feb. 25; 21(4):787-802; Ito et al. Nucleic Acids Res. 1991 Aug. 11; 19(15):4045-57; Sambrook & Russell, Molecular Cloning: A Laboratory Manual 7.75-8.126, A4.11-A4.29 (3d Cold Spring Harbor Laboratory Press (ISBN: 0879695773)).

In addition to E. coli DNA polymerase I, other non-limiting examples of A family polymerases include Bacillus, Rhodothermus, Thermotoga (e.g., Thermotoga maritima (ULTma™, New England Biolabs, Beverly, Mass.), Streptococcus pneumonia, Thermus aquaticus (e.g., Taq, Amplitaq® and Thermus flavus (e.g., HOT TUB™, Pyrostase™), Thermus thermophilus (e.g., Tth) DNA polymerases; T5, T7, SPO1, and SPO2 bacteriophage DNA polymerases; and yeast mitochondrial DNA polymerase (MIPI). (Akhmetzjanov et al. Nucleic Acids Res. 1992 Nov. 11; 20(21):5839; Al-Soud et al., Appl Env Micro. 1998; 64:3748; Blanco et al. Nucleic Acids Res. 1991 Feb. 25; 19(4):955; Dunn et al. J Mol Biol. 1983 Jun. 5; 166(4):477-535; Foury et al. J Biol Chem. 1989 Dec. 5; 264(34):20552-60; Hahn et al. Nucleic Acids Res. 1989 Aug. 25; 17(16):6729; Hollingsworth et al. J Biol Chem. 1991 Jan. 25; 266(3):1888-97; Ito et al. Nucleic Acids Res. 1990 Nov. 25; 18(22):6716; Johnson et al. J Biol Chem. 2003; 278:23762; Joyce et al. J Biol Chem. 1982 Feb. 25; 257(4):1958-64; Kaliman et al. FEBS Lett. 1986 Jan. 20; 195(1-2):61-4; Lawyer et al. J Biol Chem. 1989 Apr. 15; 264(11):6427-37; Leavitt et al. Proc Natl Acad Sci U S A. 1989 June; 86(12):4465-9; Raden et al. J Virol. 1984 October;52(1):9-15; Scarlato et al. Gene. 1992 Sep. 1; 118(1):109-13; Yehle et al. J Biol Chem. 1973; 248:7456-7463).

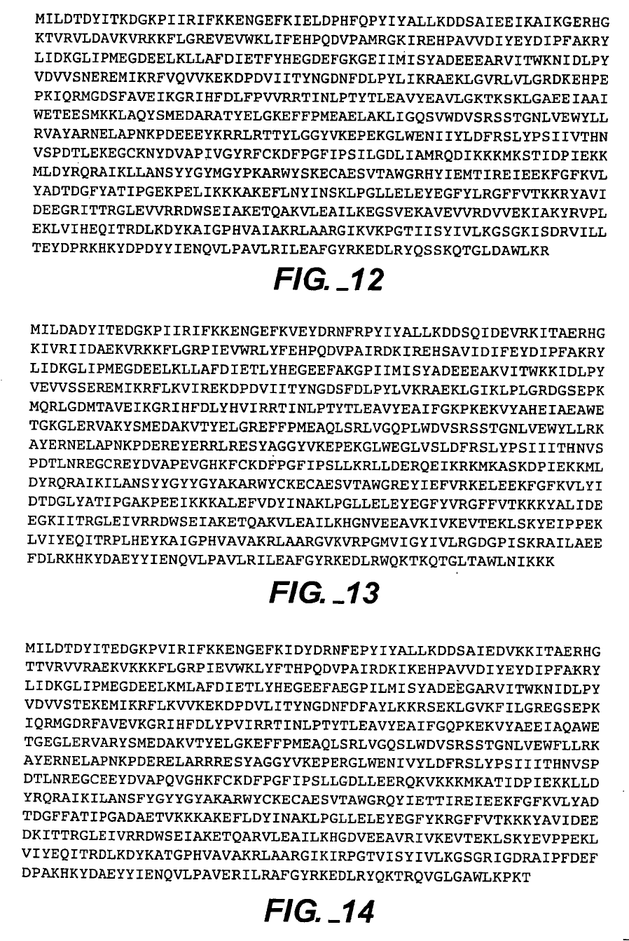

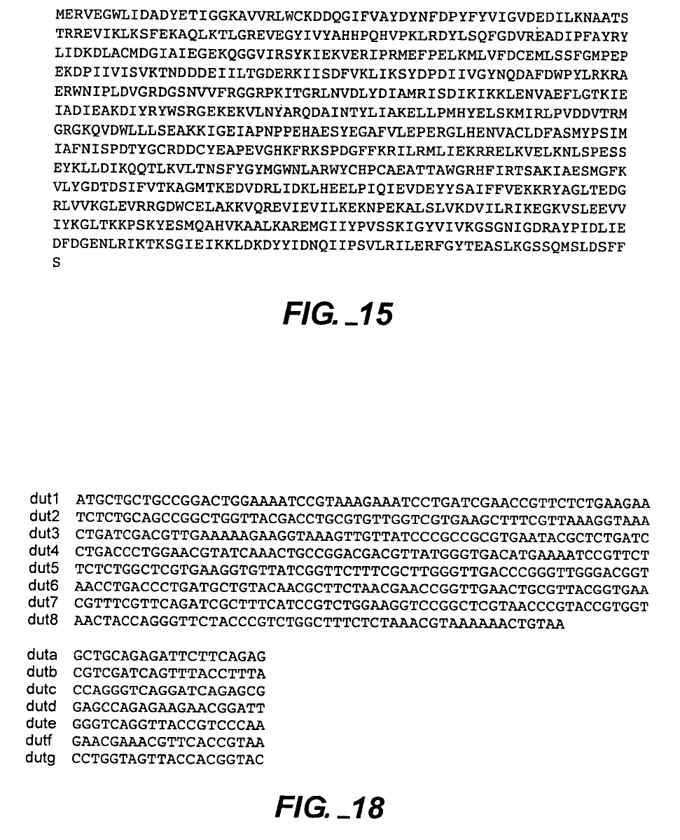

Examples of B family DNA polymerases include E. coli DNA polymerase II; PRD1, φ29, M2, and T4 bacteriophage DNA polymerases; archaebacterial DNA polymerase I (e.g. Thermococcus litoralis (Vent™, GenBank: AAA72101, FIG. 12), Pyrococcus furiosus (Pfu, GenBank: D12983, BAA02362, FIG. 10), Pyrococcus GB-D (Deep Vent™, GenBank: AAA67131, FIG. 13), Thermococcus kodakaraensis KODI (KOD, GenBank: BD175553, FIG. 11; Thermococcus sp. strain KOD (Pfx, GenBank: AAE68738)), Thermococcus gorgonarius (Tgo, GenBank: P56678, 029753, FIG. 14), Sulfolobus solataricus (GenBank: NC_{—}002754), Aeropyrum pernix (GenBank: BAA81109), Archaeglobus fulgidus (GenBank: 029753, FIG. 15), Pyrobaculum aerophilum (GenBank: AAL63952), Pyrodictium occultum (GenBank: B56277), Thermococcus 9° Nm (GenBank: AAA88769), Thermococcus fumicolans (GenBank: CAA93738), Thermococcus gorgonarius (Tgo, GenBank: P56689), Thermococcus hydrothermalis (GenBank: CAC18555), Thermococcus spp. GE8 (GenBank: CAC12850), Thermococcus spp. JDF-3 (GenBank: AX135456; WO0132887), Thermococcus spp. TY (GenBank: CAA73475), Pyrococcus abyssi (GenBank: P77916), Pyrococcus glycovorans (GenBank: CAC12849), Pyrococcus horikoshii (GenBank: NP 143776), Pyrococcus spp. GE23 (GenBank: CAA90887), Pyrococcus spp. ST700 (GenBank: CAC12847), Desulfurococcus, Pyrolobus, Pyrodictium, Staphylothermus, Vulcanisaetta, Methanococcus (GenBank: P52025) and other archael B polymerases, such as GenBank AAF27815, AAC62712, P956901, P26811, BAAA07579)); human DNA polymerase (α), S. cerevisiae DNA polymerase I (α), S. pombe DNA polymerase I (α), Drosophila melanogaster DNA polymerase (α), Trypanosoma brucei DNA polymerase (α), human DNA polymerase (δ), bovine DNA polymerase (δ), S. cerevisia DNA polymerase III (δ), S. pombe DNA polymerase III (δ), P. falciparum DNA polymerase (δ), S. cerevisiae DNA polymerase II (δ), S. cerevisiae DNA polymerase Rev3; viral DNA polymerases of herpes simplex I, equine herpes virus I, varicella-zoster virus, Epstein-Barr virus, Herpesvirus saimiri, human cytomegalovirus, murine cytomegalovirus, human herpes virus type 6, channel catfish virus, chlorella virus, fowlpox virus, vaccinia virus, Choristoneura biennis entomopoxvirus, Autographa califomica nuclear polyhydedrosis virus (AcMNPV), Lymantria dispar nuclear polyhedrosis virus, adenovirus-2, adenovirus-7, adenovirus-12; and eukaryotic linear DNA ploasmid encoded DNA polymerases (e.g., S-1 maize, Kalilo neurospora intermedia, pA12 Ascobolus immersus, pCLK1 Claviceps purpurea, maranhar neurospora crassa, pEM Agaricus bitorquis, pGLK1 Kluveromyces lactis, pGKL2 Kluveromyces lactis, and pSKL Saccharomyces kluyveri. (Albrecht et al. Virology. 1990 February; 174(2):533-42; Baer et al. DNA sequence and expression of the B95-8 Epstein-Barr virus genome. Nature. 1984 Jul. 19-25; 310(5974):207-11; Binns et al. Nucleic Acids Res. 1987 Aug. 25; 15(16):6563-73; Bjornson et al. J Gen Virol. 1992 June; 73 (Pt 6):1499-504. Erratum in: J Gen Virol 1994 December; 75(Pt 12):3687; Chan et al. Curr Genet. 1991 August; 20(3):225-37; Chung et al. Proc Natl Acad Sci USA. 1991 Dec. 15; 88(24):11197-201; Court et al. Curr Genet. 1992 November; 22(5):385-97; Damagnez et al. Mol Gen Genet. 1991 April; 226(1-2):182-9; Davison et al. Virology. 1992 January; 186(1):9-14; Davison et al. J Gen Virol. 1986 September; 67 (Pt 9):1759-816.; Earl et al. Proc Natl Acad Sci USA. 1986 June; 83(11):3659-63; Elliott et al. Virology. 1991 November; 185(1):169-86; Engler et al. Gene. 1983 January-February; 21(1-2):145-59; Gibbs et al. Proc Natl Acad Sci USA. 1985 December; 82(23):7969-73; Gingeras et al. J Biol Chem. 1982 Nov. 25; 257(22):13475-91; Grabherr et al. Virology. 1992 June; 188(2):721-31; Hirose et al. Nucleic Acids Res. 1991 Sep. 25; 19(18):4991-8; Hishinuma et al. Mol Gen Genet. 1991 April; 226(1-2):97-106; Iwasaki et al. Mol Gen Genet. 1991 April; 226(1-2):24-33; Jung et al. Proc Natl Acad Sci USA. 1987 December; 84(23):8287-91; Kempken et al. Mol Gen Genet. 1989 September; 218(3):523-30; Konisky et al., J Bacteriol. 1994; 176(20):6402-6403; Kouzarides et al. J Virol. 1987 January; 61(1):125-33; Leegwater et al. Nucleic Acids Res. 1991 Dec. 11; 19(23):6441-7; Matsumoto et al. Gene. 1989 Dec. 14; 84(2):247-55; Mustafa et al. DNA Seq. 1991; 2(1):39-45; Morrison et al. Cell. 1990 Sep. 21; 62(6):1143-51; Morrison et al. J Bacteriol. 1989 October; 171(10):5659-67; Morrison et al. Nucleic Acids Res. 1992 Jan. 25; 20(2):375; Nishioka et al. J Biotechnol. 2001; 88:141-149; Oeser et al. Mol Gen Genet. 1989 May; 217(1):132-40; Paillard et al. EMBO J. 1985; 4:1125-1128; Perler et al. Proc Natl Acad Sci USA 1992 Jun. 15; 89(12):5577-81; Pignede et al. J Mol Biol. 1991 Nov. 20; 222(2):209-18. Erratum in Pisani et al. Nucleic Acids Res. 1992 Jun. 11; 20(11):2711-6; Pizzagalli et al. Proc Natl Acad Sci USA. 1988 June; 85(11):3772-6; Robison et al. Curr Genet. 1991 June; 19(6):495-502; Savilahti et al. Gene. 1987; 57(1):121-30; Shu et al. Gene. 1986; 46(2-3):187-95; Spicer et al. J Biol Chem. 1988 Jun. 5; 263(16):7478-86; Stark et al. Nucleic Acids Res. 1984 Aug. 10; 12(15):6011-30.; Takagi et al. Appl Environ Microbiol. 1997; 63:4505-4510; Telford et al. Virology. 1992 July; 189(1):304-16; Teo et al. J Virol. 1991 September; 65(9):4670-80; Tomalski et al. Virology. 1988 December; 167(2):591-600; Tommasino et al. Nucleic Acids Res. 1988 Jul. 11; 16(13):5863-78; Wong et al. EMBO J. 1988 January; 7(1):37-47; Yang et al. Nucleic Acids Res. 1992 Feb. 25; 20(4):735-45; Yoshikawa et al. Gene. 1982 March; 17(3):323-35)

Examples of type C family DNA polymerases include DNA polymerase III of E. coli (α), S. typhimirium (α), Bacillus subtilis, and E. coli dnaQ (MutD) (E. coli DNA polymerase III (ε)). (Hammond et al. Gene. 1991 Feb. 1; 98(1):29-36; Joyce et al. (1986) In “Protein Structure, Folding and Design (UCLA Symposia on Molecular and Cellular Biology, Vol. 32), D. Oxender, Ed., pp. 197-205, Alan R. Liss; Lancy et al. J Bacteriol. 1989 October; 171(10):5581-6. Erratum in: J Bacteriol 1991 July; 173(14):4549; Maki et al. Proc Natl Acad Sci USA. 1983 December; 80(23):7137-41).

“dUTPase domain” as used herein refers to an amino acid sequence having deoxyuridine triphosphate nucleotidehydrolase activity (dUTPase, e.g., EC 3.6.1.23) Therefore, a dUTPase domain can hydrolyze dUTP to dUMP and pyrophosphate. In various exemplary embodiments, a dUTPase domain can comprise all of part of the amino acid sequence of a dUTPase. dUTPases are ubiquitous and can be isolated from various cells and organisms. In some embodiments, a dUTPase domain can be thermostable. Sources of amino acid sequences comprising dUTPase activity include but are not limited to eukaryotic cells (e.g., plant, human (e.g., nuclear and mitochondrial isoforms), murine, yeast (e.g., Candida, Saccharomyces) and protozoa (e.g., Leishmania), prokaryotic cells (e.g., eubacteria (e.g., E. coli) and archaebacteria (e.g., Pyrococcus, Aeropyrum, Archaeglobus, Pyrodictium, Sulfolobus, Thermococcus Desulfurococcus, Pyrobaculum, Pyrococcus, Staphylothermus, Stetteria, Sulfolobus, Thermococcus, and Vulcanisaeta) and viruses (e.g., bacteriophages (e.g., T5), poxviruses (e.g. vaccinia virus, African swine fever viruses), retroviruses (e.g., lentiviruses, equine infectious anemia virus, mouse mammary tumor virus), herpesviruses, nimaviruses (e.g., Shrimp white spot syndrome virus), endogenous retroviruses (e.g., HERV-K), and archaeal viruses (SIRV). (Baldo et al. J Virol. 1999 September; 73(9):7710-21; Barabas et al. J Biol Chem. 2003 Oct. 3; 278(40):38803-12. Epub 2003 Jul 16; Bergman et al. Protein Expr Purif. 1995 June; 6(3):379-87; Bjomberg et al. Protein Expr Purif. 1993 April; 4(2):149-59; Broyles. Virology. 1993 August; 195(2):863-5; Camacho et al. Biochem J. 1997 Jul. 15; 325 (Pt 2):441-7; Camacho et al. Biochem J. 1997 Jul. 15; 325 ( Pt 2):441-7; Caradonna et al. Curr Protein Pept Sci. 2001 December; 2(4):335-47; Caradonna et al. J Biol Chem. 1984 May 10; 259(9):5459-64; Cottone et al. J Gen Virol. 2002; 83:1043; Chakravarti et al. J Biol Chem. 1991 Aug. 25; 266(24):15710-5; Chu et R, Lin Y, Rao M S, Reddy J K. J Biol Chem. 1996 Nov. 1; 271(44):27670-6; Cohen et al. Genomics 40: 213-215, 1997; Dabrowski et al. Protein Expr Purif. 2003 September; 31(1):72-8; Doignon et al. Yeast. 1993 October; 9(10):1131-7; Elder et al. J Virol. 1992 March; 66(3):1791-4; Engelward et al. Carcinogenesis. 1993 February; 14(2):175-81; Fiser et al. Biochem Biophys Res Commun. 2000 Dec. 20; 279(2):534-42; Flowers et al. Proc Natl Acad Sci U S A. 1995 May 9; 92(10):4274-8; Hanash et al. Proc Natl Acad Sci U S A. 1993 Apr. 15; 90(8):3314-8; Harris et al. Biochem Cell Biol. 1997; 75(2):143-51; Jons et al. J Virol. 1996 February; 70(2):1242-5; Kaliman. DNA Seq. 1996; 6(6):347-50; Kan et al. Gene Expr. 1999; 8(4):231-46; Koppe et al. J Virol. 1994 April; 68(4):2313-9; Kovari et al. Nucleosides Nucleotides Nucleic Acids. 2004 October; 23(8-9):1475-9; Ladner et al. J Biol Chem. 1996 Mar. 29; 271(13):7745-51; Ladner et al. J Biol Chem. 1996 Mar. 29; 271(13):7752-7; Ladner et al. J Biol Chem. 1997 Jul. 25; 272(30):19072-80; Ladner et al. Cancer Res. 2000 Jul. 1; 60(13):3493-503; Liang et al. Virology. 1993 July; 195(1):42-50 ; Liu et al. Virus Res. 2005 June; 110(1-2):21-30; Lundberg et al. EMBO J. 1983; 2(6):967-71; Mayer et al. J Mol Evol. 2003 December; 57(6):642-9; McGeehan et al. Curr Protein Pept Sci. 2001 December; 2(4):325-33; McIntosh et al. Curr Genet. 1994 November-December; 26(5-6):415-21. Erratum in: Curr Genet 1995 April; 27(5):491; McIntosh et al. Proc Natl Acad Sci USA. 1992 Sep. 1; 89(17):8020-4. Erratum in: Proc Natl Acad Sci USA 1993 May 1; 90(9):4328; Miyazawa et al. J Biol Chem. 1993 Apr. 15; 268(11):8111-22; Oliveros et al. J Virol. 1999 November; 73(11):8934-43; Persson et al. Curr Protein Pept Sci. 2001 December; 2(4):287-300; Persson et al. Prep Biochem Biotechnol. 2002 May; 32(2): 157-72; Prangishvili et al. J Biol Chem. 1998 Mar. 13; 273(11):6024-9; Prasad et al. Protein Sci. 1996 December; 5(12):2429-37; Pri-Hadash et al. Plant Cell. 1992 February; 4(2):149-59; Shao et al. Biochim Biophys Acta. 1997 May 23; 1339(2):181-91; Spector et al. J Neurochem. 1983 October; 41(4):1192-5; Strahler et al. Proc Natl Acad Sci USA. 1993; 90:4991-4995; Threadgill et al. J Virol. 1993 May; 67(5):2592-600; Turelli et al. J Virol. 1996 February; 70(2):1213-7; Weiss et al. J Virol. 1997 March; 71(3):1857-70).

“Processivity domain” as used herein refers to a sequence suitable for increasing the processivity of the polymerase. Generally, processivity domains comprise sequences with an affinity for non-specific or sequence independent binding to DNA. Without being bound by theory, improved processivity can be hypothesized to operate by increasing the affinity of the chimeric polymerase for DNA. In various exemplary embodiments, processivity domains can comprise a double-stranded DNA binding protein sequence (WO01/92501), a helix-turn-helix (HTH) motif sequence, such as found in topoisomerase V from Methanopyrus kandleri (Pavlov et al. Proc Natl Acad Sci USA. 2002; 99:13510-13515), PCNA-like protein sequence (see, e.g., U.S. Pat. No. 6,627,424; Bedford et al. Proc Natl Acad Sci USA. 94:479-484).

“Double-stranded DNA binding protein (dsDBP)” and “nucleic acid binding protein” as used herein refers to a protein or a subsequence or fragment thereof that binds to double-stranded DNA in a sequence independent manner, i.e., binding does not exhibit a substantial preference for a particular sequence. Typically, dsDBP exhibit at least about a 10-fold or higher affinity for double-stranded versus single-stranded polynucleotides. In some embodiments, dsDBP can be thermostable.

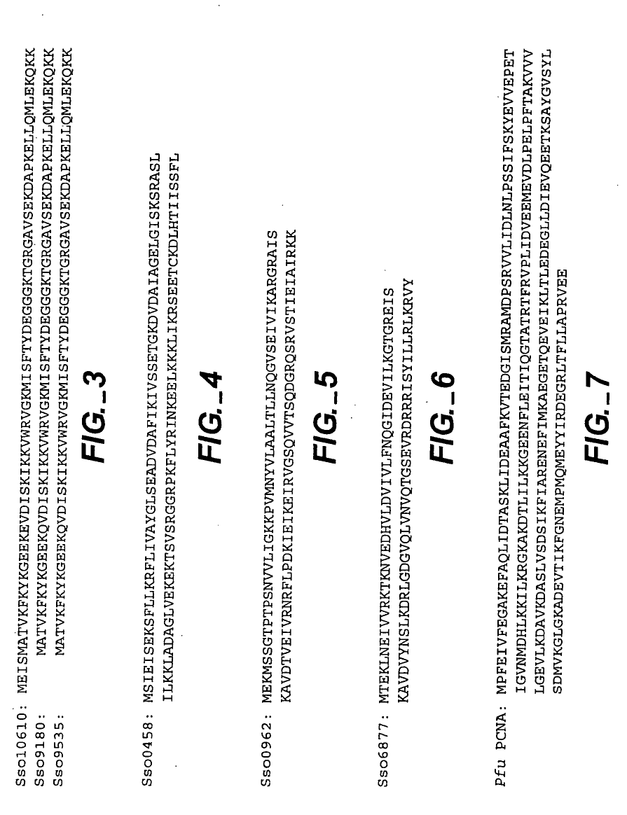

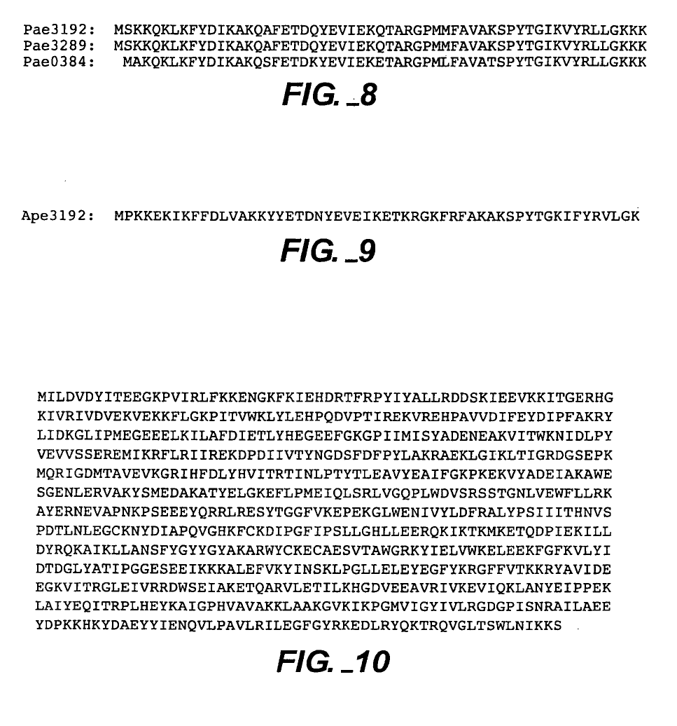

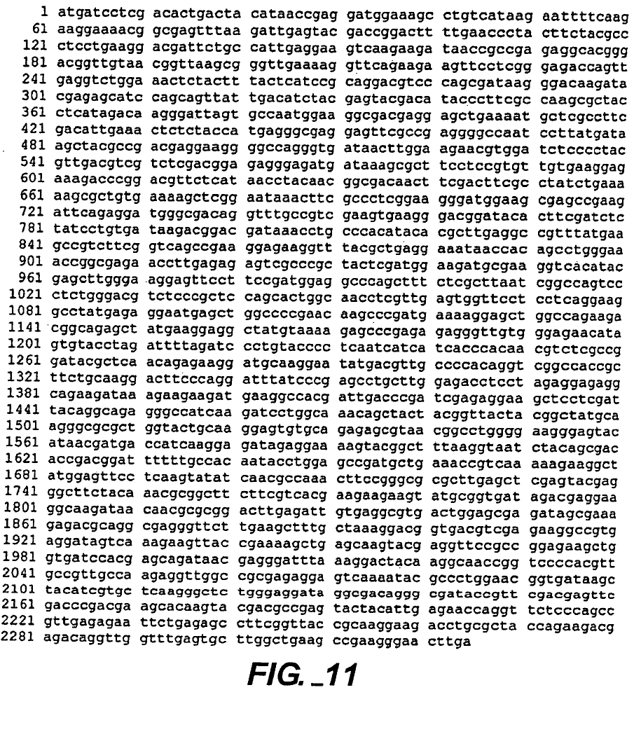

Archaeal dsDBP generally are generally small (˜7Kd), basic chromosomal proteins that are lysine-rich and have high thermal, acid and chemical stability. They bind DNA in a sequence-independent manner and when bound, increase the Tₘ of DNA by up to about 40° C. (McAfee et al., Biochemistry 1995; 34:10063-10077; Robinson et al. Nature 1998; 392:202-205). Examples of such proteins include, but are not limited to, the Archaeal DNA binding proteins Ape3192 (FIG. 9), Pae3l92, Pae3289, Pae0384, (FIG. 8), Sac7d, Sso7d (FIG. 3) (Choli et al. Biochimica et Biophysica Acta 1988; 950:193-203; Baumann et al., Structural Biol. 1994; 1:808-819; Gao et al. Nature Struc. Biol. 1998; 5:782-786, 1998; Wang et al. Nuc Acids Res. 2004; 32:1197-1207), Smj12 (FIG. 4) (Napoli et al. J Biol Chem. 2001 Apr. 6; 276(14):10745-52. Epub 2001 Jan. 8), Alba-1 (Sso10b-1, Sac10a) (FIG. 5) (Wardleworth et al. EMBO J. 2002 Sep. 2; 21(17):4654-62); Alba-2 (Sso6877) (FIG. 6) (Chou et al. J Bacteriol. 2003; 185:4066-4073); Archaeal HMf-like proteins (Starich et al., J. Molec. Biol. 1996; 255:187-203; Sandman et al., Gene 1994; 150:207-208), and PCNA homologs (FIG. 7) (Cann et al., J. Bacteriology 1999; 181:6591-6599; Motz et al. J Biol Chem. 2002 May 3; 277(18):16179-88. Epub 2002 Jan. 22; Shamoo and Steitz, Cell:99, 155-166, 1999; De Felice et al., J. Molec. Biol. 291, 47-57, 1999; Zhang et al., Biochemistry 34:10703-10712, 1995).

Three copies of Sso7d and its direct paralogs (Sso10710, Sso9180, Sso9535) can be found in the genome of S. sulfataricus P2. (She et al. Proc Natl Acad Sci USA. 2001 Jul. 3; 98(14):7835-40. Epub 2001 Jun. 26). Sso1016 is a generic name for ORF 10610 of S. sulfataricus P2, and the number, 10610, is a linear designation to reflect its position on the circular chromosome relative to “1” which is frequently chosen as the origin or replication. As shown in FIG. 3, these three paralogs are almost completely identical and are thought to have arisen as a result of gene duplications.

ORFs encoding Pae3192, Pae3299, and Pae0384 can be found in the genome of the Crenarchaeote Pyrobaculum aerophilum strain IM2. As shown in FIG. 8, these sequences of these proteins also are similar and may have arisen by gene duplication. In the genome of P. aerophilum (GenBank AE009441, NC_{—}003364), the “Pae” ORFS are designated paREP4.

An ORF encoding Ape3192 can found in a non-annotated region of the genome of Aeropyrum pernix (GenBank NC_{—}000854) by amino acid sequence homology to Pae3192.

HMf-like proteins are archaeal histones that share homology both in amino acid sequence and in structure with eukaryotic H4 histones. The HMf family of proteins form stable dimers in solution, and several HMf homologs have been identified from thermophilic organisms (e.g., Methanothermus fervidus and Pyrococcus ssp. GB-3a). The HMf family of proteins, once joined to DNA polymerase can enhance the ability of the enzyme to slide along the DNA substrate and thus increase its processivity.

Many B-family DNA polymerases interact with accessory proteins to achieve highly processive DNA synthesis. Once class of accessory proteins can be referred to as the sliding clamp. Several characterized sliding clamps exist as trimers in solution, and can form a ring-like structure with a central passage capable of accommodating double-stranded DNA. The sliding clamp can form specific interactions with the amino acids located at the carboxy terminus of particular DNA polymerases, and tethers those polymerases to the DNA template during replication. The sliding clamp in eukarya is referred to as the proliferating cell nuclear antigen (PCNA), while similar proteins in other domains are often referred to as PCNA homologs (e.g., dnaN-like or PCNA-like). PCNA homologs have been identified from thermophilic Archaea (e.g., Archaeoglobis fulgidis, Sulfolobus sofataricus, Pyroccocusfuriosus, etc.) (Motz et al J Biol Chem. 2002; 277:16179-16188). Some B-family polymerases in Archaea have a carboxy terminus containing a consensus PCNA-interacting amino acid sequence and are capable of using a PCNA homolog as a processivity factor (Cann et al., J. Bacteriol. 1999; 181:6591-6599; De Felice et al., J. Mol. Biol. 1999; 291:47-57, 1999). PCNA homologs can be useful as sequence-non-specific double-stranded DNA binding domains that can be fused to a polymerizing domain. For example, a consensus PCNA-interacting sequence can be joined to a polymerase that does not naturally interact with a PCNA homolog, thereby allowing a PCNA homolog to serve as a processivity factor for the polymerase.

In some embodiments, a chimeric polymerases comprises a sequence that includes a variant (e.g., mutant or fragment) of a naturally occurring polypeptide sequence. In various exemplary embodiments, the variant sequence has from about 60%, 65%, 70%, 75%, 80%, 85%, 90%, 95%, 96%, 97%, 98% to about 99% identity to a naturally occurring sequence. In some embodiments, the identity is at least about 95%. In various exemplary embodiments, a variant sequence can have 0%, 10%, 20%, 30%, 40%, 50%, 60%, 70%, 80%, 90%, 100%, or >100% activity of a naturally occurring polypeptide sequence.

In some embodiments, a chimeric polymerase can comprise one or more mutations suitable for increasing or decreasing one or more activities or properties of a chimeric polymerase. For example, in some embodiments, a chimeric polypeptide comprising an Archael B-family DNA polymerizing domain can comprise one or more mutations suitable for substantially inactivating the base-analog detection or read-ahead domain. “Base analog detection domain” or “read-ahead domain” as used herein refers to an amino acid sequence that is capable of detecting one or more base analogs in a DNA template. (Greagg et al. Proc Natl Acad Sci USA. 1999; 96:9045-50). “Base analog” refers to bases other than adenine, thymine, guanine, and cytosine that can be present in DNA. In some embodiments, a base analog can be a naturally-occurring base analog, such as, uracil or inosine which can be generated by deamination of cytosine or adenine, respectively. In some embodiments, a base analog can be a non-naturally occurring base analog, including but not limited to 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines, (see, e.g., Held et al. Nucl Acids Res. 2002; 30:3869; U.S. Pat. Nos. 6,143,877, 6,127,121; U.S. Patent Application Nos. 2004091873, 20040086890, 20040081965, 20050069908, 20040009486, 20030157483, and PCT published applications WO2004/03807; WO01/38584), ethenoadenine, indoles such as nitroindole and 4-methylindole, and pyrroles such as nitropyrrole. Certain exemplary nucleotide bases can be found, e.g., in Fasman (1989) Practical Handbook of Biochemistry and Molecular Biology, pages 385-394, (CRC Press, Boca Raton, Fla.) and the references cited therein. Examples of mutations suitable for substantially reducing base analog detection include one or more mutations at one or more of the following amino acid positions corresponding to Pfu polymerase: V93Q, V93R, V93E, V93A, V93K, V93Q, V93N, V93Δ, and P115Δ. Other examples of mutations suitable for substantially reducing base analog detection include mutations at following the amino acid positions corresponding to Pfu polymerase: D92Δ, V93Δ, and P94Δ.

In some embodiments, mutations suitable for substantially reducing base-analog detection can reduce the specific activity of chimeric polymerases by up to about 50%. In some embodiments, chimeric polymerases comprising one or more processivity domains can at least partially offset this loss of specific activity. In some embodiments, chimeric polymerases comprising mutations at one or more amino acid positions corresponding to Pfu polymerase can be introduced to offset this loss of specific activity (e.g., M247R, T265R, K502K, A408S, K485R, L381Δ). (FIG. 16). In various exemplary embodiments, at least about 1%, 5%, 10%, 15%, 20%, 25%, 30%, 35%, 40%, 45%, 50%, 55%, 60%, 65%, 70%, 75%, 80%, 85%, 90%, 95%, 99%, 100%, and greater than 100% activity can be restored.

In some embodiments, mutations suitable for substantially reducing the 3′→5′ exonuclease activity of an Arachaeal B-family polymerase can be made at a consensus “DIET” (SEQ ID NO:81) motif (corresponding to amino acids 141-144 of Pfu polymerase). In some embodiments, the consensus motif can be mutated, for example, to “DIDT” (SEQ ID NO:82) (E143D) or “AIAT” (SEQ ID NO:83) (D141A, E143A) to either substantially reduce (e.g., ˜5-10% of normal) or abolish exonuclease activity, respectively. Other mutations that at least substantially reduce 3′→5′ exonuclease activity, either alone or in combination, include D141A, D141N, D141S, D141T, D141E, E143A, and the amino acid positions corresponding thereto in other polymerases. (U.S. Patent Application Publication No. 20050069908; Southworth et al. Proc Natl Acad Sci USA. 1996 May 28; 93(11):5281-5; Derbyshire et al. Methods Enzymol. 1995; 262:363-385; Kong et al. J Biol Chem. 1993 Jan. 25; 268(3):1965-75). In some embodiments, the amino acid corresponding to D215 of Pfu polymerase can be substituted by Ala to substantially reduce 3′→5′ exonuclease activity. Methods of determining exonuclease activity as disclosed in U.S. Patent Application Publication No. 20050069908 .

In some embodiments, mutations that allow incorporation of non-natural nucleotides/nucleotide analogs into a nascent DNA strand can be incorporated into a chimeric polymerase. In some embodiments, such mutations can be used in combination with the exonuclease mutations described above (e.g., D141A, E143A), to prevent a chimeric polymerase from excising a non-naturally occurring base analog from a nascent DNA strand. In various exemplary embodiments, these mutations that allow the incorporation of nucleotide analogs include a substitution of a Leu at a position in a chimeric polypeptide corresponding to residue Pro-410 of Pfu polymerase (P410L) and a substitution of a Thr at a position corresponding to Ala-483 of Pfu polymerase (A485T). The P410L mutation can increase the incorporation efficiency of non-naturally occurring base analogs by about 50 fold. The A485T mutation increases incorporation efficiency by about 10 fold. (Arezi et al. J Mol Biol. 2002 Sep. 27; 322(4):719-29; Gardner et al., (1999) Nucl. Acids Res. 27:2545-2555; Gardner et al. (2002) Nucl. Acids Res. 30:605-613; New England Biolabs. Technical Bulletin #M0261 (Sep. 28, 2004).

Thus, in various exemplary embodiments, the B-Pol domain as shown in FIG. 2A-E can be a polymerizing domain of Thermococcus litoralis, Pyrococcus furiosus, Pyrococcus GB-D, Thermococcus kodakaraensis KODI, Thermococcus sp. strain KOD, Thermococcus gorgonarius, Sulfolobus solataricus, Aeropyrum pernix, Archaeglobus fulgidus, Pyrobaculum aerophilum, Pyrodictium occultum, Thermococcus 9° Nm, Thermococcusfumicolans, Thermococcus hydrothermalis, Thermococcus spp. GE8, Thermococcus spp. JDF-3, Thermococcus spp. TY, Pyrococcus abyssi, Pyrococcus glycovorans, Pyrococcus horikoshii, Pyrococcus spp. GE23, Pyrococcus spp. ST700, Desulfurococcus, Pyrolobus, Pyrodictium, Staphylothermus, Vulcanisaetta, Methanococcus. As shown in FIGS. 2B, 2D, each of the exemplified B-Pol domains can be optionally fused to a BP domain which can be a double-stranded DNA binding protein sequence (WO01/92501), an HTH, a PCNA-like protein sequence, Ape3192, Pae3192, Pae3289, Pae0384, Sac7d, Sso7d, Smj12, Alba-1 (Sso10b-1, Sac10a), Alba-2 (Sso6877), Archaeal HMf-like proteins, PCNA homologs, Sso7d and its direct paralogs (Sso10710, Sso9180, Sso9535), Sso1016, Pae3299. As shown in FIGS. 2B, 2C, 2D, and 2E, a chimeric polymerase can optionally include a dUTPase domain which can be from plants, humans (e.g., nuclear and mitochondrial isoforms), mammals, yeast (e.g., Candida, Saccharomyces) and protozoa (e.g., Leishmania), prokaryotic cells (e.g., eubacteria (e.g., E. coli) and archaebacteria (e.g., Pyrococcus, Aeropyrum, Archaeglobus, Pyrodictium, Sulfolobus, Thermococcus Desulfurococcus, Pyrobaculum, Pyrococcus, Staphylothermus, Stetteria, Sulfolobus, Thermococcus, and Vulcanisaeta) and viruses (e.g., bacteriophages (e.g., T5), poxviruses (e.g. vaccinia virus, African swine fever viruses), retroviruses (e.g., lentiviruses, equine infectious anemia virus, mouse mammary tumor virus), herpesviruses, nimaviruses (e.g., Shrimp white spot syndrome virus), endogenous retroviruses (e.g., HERV-K), and archaeal viruses (SIRV). The chimeric polymerases exemplified in FIG. 2 optionally contain one or more mutations that decrease base analog detection, such as, one or more mutations at one or more of the following amino acid positions corresponding to Pfu polymerase: V93Q, V93R, V93E, V93A, V93K, V93Q, V93N, V93G, V93Δ, P115Δ, D92Δ, and P94Δ. The chimeric polymerases exemplified in FIG. 2 optionally include mutations that increase the specific activity of the chimeric polymerase such as mutations corresponding to Pfu polymerase: M247R, T265R, K502K, A408S, K485R, L381Δ. In some embodiments, the chimeric polymerases exemplified in FIG. 2 optionally include a 3′→5′ exonuclease domain. In some embodiments, a 3′→5′ exonuclease domain, if present, can be substantially activated by the optional introduction of one or more mutations at amino acids corresponding to Pfu polymerase: E143D, D141A, E143A, D141A, D141N, D141S, D141T, D141E, E143A, D215A. In some embodiments, the chimeric polymerases exemplfied in FIG. 2 optionally include one or more mutations that allow incorporation of non-natural nucleotides/nucleotide analogs into a nascent DNA strands, such as, mutations at amino acids corresponding to P410L and A485T.

The various domains of the chimeric polypeptides disclosed herein can be can be joined and mutations can be introduced by methods well known to those of skill in the art, such as, chemical and recombinant methods.

Methods of chemically joining heterologous domains are described, e.g., in Bioconjugate Techniques, Hermanson, Ed., Academic Press (1996). These include, for example, derivitization for the purpose of linking domains, either directly or through a linking compound, by methods that are well known in the art of protein chemistry. For example, in some embodiments, a linker can comprise a heterobifunctional coupling reagent which ultimately contributes to formation of an intermolecular disulfide bond between the domains. Other types of coupling reagents that are useful in this capacity are described, for example, in U.S. Pat. No. 4,545,985. Alternatively, an intermolecular disulfide can be formed between cysteines in each domain, which occur naturally or are introduced by recombinant DNA techniques. Domains also can be linked using thioether linkages between heterobifunctional crosslinking reagents or specific low pH cleavable crosslinkers or specific protease cleavable linkers or other cleavable or noncleavable chemical linkages.

In some embodiments, heterologous domains can be joined by a peptidyl bond formed between domains that can be separately synthesized by standard peptide synthesis chemistry or recombinant methods. A chimeric polypeptide can also be produced in whole or in part using chemical methods. For example, in some embodiments, peptides can be synthesized by solid phase techniques, such as, the Merrifield solid phase synthesis method (J. Am. Chem. Soc. 1963; 85:2149-2146). The synthesized peptides can then be cleaved from the resin, and purified by one or more methods as known in the art. (Creighton, Proteins Structures and Molecular Principles, 1983; 50-60). The composition of the synthetic polypeptides may be confirmed by amino acid analysis or sequencing (Creighton, Proteins, Structures and Molecular Principles 1983; pp. 34-49).

In some embodiments, a chimeric polymerase can comprise one or more amino acid analogs. Examples of amino acid analogs include, but are not limited to, D-isomers of the common amino acids, α-amino isobutyric acid, 4-aminobutyric acid, 2-amino butyric acid, 6-amino hexanoic acid, 2-amino isobutyric acid, 3-amino propionic acid, omithine, norleucine, norvaline, hydroxy-proline, sarcosine, citrulline, cysteic acid, t-butylglycine, t-butylalanine, phenylglycine, cyclohexylalanine, β-alanine, fluoroamino acids, β-methyl amino acids, and α-methyl amino acids. Furthermore, the amino acid can be D (dextrorotary) or L (levorotary). In various exemplary embodiments, amino acid analogs can be introduced before and/or after joining one or more domains of the chimeric polymerase.

In some embodiments, the domains of a chimeric polypeptide can be joined via a linker, such as, a chemical crosslinking agent (e.g., succinimidyl-(N-maleimidomethyl)-cyclohexane-1-carboxylate (SMCC)). The linking group can also comprise one or more amino acid sequence(s), including, for example, a polyalanine, polyglycine, and the like.

In some embodiments, coding sequences of each domain of a chimeric polypeptide can be directly joined at their amino- or carboxy-terminus via a peptide bond in any order. Alternatively, an amino acid linker sequence may be employed to separate the domains. In some embodiments, such linker sequence can be used to promote proper folding of the chimeric polymerase. Such an amino acid linker sequences can be incorporated into the chimeric polypeptide using standard techniques well known in the art. Suitable peptide linker sequences may be chosen based on the following factors, including but not limited to: (1) their ability to adopt a flexible extended conformation; (2) their inability to adopt a desired secondary or tertiary structure; and (3) the presence or absence of hydrophobic, charged and/or polar residues. Non-limiting examples of peptide linker sequences contain Gly, Val, Ser, Ala and/or Thr residues. Exemplary amino acid sequences which may be employed as linkers include those disclosed in Maratea et al. Gene 1985; 40:39-46; Murphy et al. Proc. Natl. Acad. Sci USA. 1986; 83:8258-8262; U.S. Pat. Nos. 4,935,233 and 4,751,180. In various exemplary embodiments, a linker sequence may generally be from about 1, 2, 3, 4, 5, 6, 7, 8, 9, 10, 15, 20, 25, 30, 35, 40, 45 to about 50 amino acids in length but can be about 100 to about 200 amino acids in length or higher.

Other methods of making chimeric polypeptides include ionic binding by expressing negative and positive tails on the various domains, indirect binding through antibodies and streptavidin-biotin interactions. The domains may also be joined together through an intermediate interacting sequence. For example, a consensus PCNA-interacting sequence can be joined to a polymerase that does not naturally interact with a PCNA homolog. The resulting fusion protein can then be allowed to associate non-covalently with the PCNA homolog to generate a novel heterologous protein with increased processivity.

In some embodiments, a chimeric polypeptide can be produced by recombinant expression of the encoding polynucleotide sequence, including linker sequences, as known in the art. Polynucleotide sequences encoding the various domains and linker sequence can be ligated in-frame and operatively linked to various constitutive or inducible promoters as known in the art. (Amann et al. (1983) Gene 25: 167; de Boer et al. (1983) Proc. Nat'l. Acad. Sci USA. 80:21; Sudier et al. (1986) J. Mol. Biol.; Tabor et al. (1985) Proc. Nat'l. Acad. Sci USA. 82: 1074-8; Gene Expression Systems, Femandex and Hoeffler, Eds. Academic Press, 1999). Polynucleotides encoding the domains to be incorporated into chimeric polypeptides can be obtained using routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

In some embodiments, polynucleotide sequences can be obtained from cDNA and genomic DNA libraries by hybridization with probes, or isolated using amplification techniques with oligonucleotide primers. Amplification techniques can be used to amplify and isolate sequences from DNA or RNA (see, e.g., Dieffenfach et al., PCR Primers: A Laboratory Manual (1995)). In some embodiments, overlapping oligonucleotides can be produced synthetically and ligated to produce one or more polynucleotides encoding one or more domains. In some embodiments, polynucleotides encoding one or more domains can also be isolated from expression libraries.

In some embodiments, a polynucleotide encoding a domain can be obtained by PCR using forward and reverse primers optionally containing one or more unique restriction enzymes to facilitate cloning. Therefore, the amplified polynucleotide sequence can be restriction enzyme digested and ligated into a vector selected at the discretion of the practitioner. In various exemplary embodiments, domains can be directly joined or may be separated by a linker, or other, protein sequence. Suitable PCR primers can be determined by one of skill in the art using the sequence information provided in GenBank or other sources (U.S. Pat. No. 4,683,202; PCR Protocols A Guide to Methods and Applications (Innis et al., eds) Academic Press Inc. San Diego, Calif. (1990); Arnheim & Levinson (Oct. 1, 1990) C&EN 36-47; The Journal Of NIH Research (1991) 3: 81-94; (Kwoh et al. (1989) Proc. Natl. Acad. Sci USA. 86: 1173; Guatelli et al. (1990) Proc. Natl. Acad. Sci USA. 87, 1874; Lomell et al. (1989) J. Clin. Chem., 35:1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117).

Recombinant vectors and host cells suitable for producing chimeric polypeptides are well known to those of ordinary skill in the art. (see, e.g., Gene Expression Systems, Fernandex and Hoeffler, Eds. Academic Press, 1999.) Typically, the polynucleotide that encodes the chimeric polypeptide can be placed under the control of a promoter that is functional in the desired host cell. Generally, the promoter selected depends upon the host cell in which the chimeric polypeptide is to be expressed. Other expression control sequences such as ribosome binding sites, transcription termination sites and the like can be optionally included.

Non-limiting examples of prokaryotic control sequences, which can include promoters for transcription initiation and an optional operator and ribosome binding site sequences, include such promoters as the beta-lactamase (penicillinase) and lactose (lac) promoter systems (Change et al., Nature (1977) 198: 1056), the tryptophan (trp) promoter system (Goeddel et al., Nucleic Acids Res. (1980) 8:4057), the tac promoter (DeBoer et al., Proc. Natl. Acad. Sci. U.S.A. (1983) 80:21-25); and the lambda-derived P_{L} promoter and N-gene ribosome binding site (Shimatake et al., Nature (1981) 292: 128). Promoters suitable for use in host cells other than E. coli include but are not limited to the hybrid trp-lac promoter finctional in Bacillus in addition to E. coli. These and other suitable promoters well known in the art and are described, e.g., in Sambrook et al., Ausubel et al., Palva et al., Gene 22:229-235 (1983); Mosbach et al., Nature 302:543-545 (1983). Non-limiting examples of bacterial expression vectors include plasmids such as pBR322-based plasmids, e.g., pBLUESCRIP™, pSKF, pET23D, λ-phage derived vectors, and fusion expression systems such as GST and LacZ. Expression vectors can optionally provide sequences encoding one or more “tags” which can be incorporated into the expressed chimeric polymerase and function to facilitate isolation and purification of the chimeric polymerase. Non-limiting examples of such tags include c-myc, HA-tag, His-tag, maltose binding protein, VSV-G tag, anti-DYKDDDDK (SEQ ID NO:76) tag, and the like.

Eukaryotic expression systems for mammalian cells, yeast, and insect cells are well known in the art. Non-limiting examples include Yeast Integrating plasmids (e.g., YIp5) and Yeast Replicating plasmids (the YRp series plasmids) and pGPD-2. Expression vectors containing regulatory elements from eukaryotic viruses also can be used for eukaryotic expression vectors, e.g., SV40 vectors, papilloma virus vectors, retrovirus vectors and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells. Non-limiting examples eukaryotic host cells suitable for expression of chimeric polypeptides include COS, CHO and HeLa cells lines and myeloma cell lines.

Once expressed, the chimeric polypeptides can be purified according to standard procedures known in the art, including ammonium sulfate precipitation, affinity columns, column chromatography, gel electrophoresis and the like (see, e.g., R. Scopes, Protein Purification, Springer-Verlag, N.Y. (1982), Deutscher, Methods in Enzymology Vol. 182: Guide to Protein Purification., Academic Press, Inc. N.Y. (1990)). To facilitate purification, the polynucleotides encoding the chimeric polypeptides can also include a coding sequence for an epitope or “tag” for which an affinity binding reagent is available. Examples of suitable epitopes include the myc and V-5 reporter genes; expression vectors useful for recombinant production of fusion polypeptides having these epitopes include pcDNA3.1/Myc-His and pcDNA3.1V5-His (Invitrogen, Carlsbad, Calif.). Additional expression vectors suitable for attaching a tag to the fusion proteins of the invention, and corresponding detection systems are known to those of skill in the art and in FLAG (Kodak, Rochester N.Y.)and a poly-His tag which is capable of binding to metal chelate affinity ligands. Suitable metal chelate affinity ligands that can serve as the binding moiety for a polyhistidine tag include nitrilo-tri-acetic acid (NTA) (Hochuli, E. (1990) “Purification of recombinant proteins with metal chelating adsorbents” In Genetic Engineering: Principles and Methods, J. K. Setlow, Ed., Plenum Press, N.Y.)). In various exemplary embodiments, sequences to facilitate purification can remain on the chimeric polymerase or can be optionally removed from by various methods as known in the art.

The chimeric polymerases described herein can be used in any method that utilizes a polymerase, including but not limited to PCR, such as, linear, assymetic, logrithmic, qPCR and real-time PCR (Blain & Goff, J. Biol. Chem. (1993) 5: 23585-23592; Blain & Goff, J. Virol. (1995) 69:4440-4452; Sellner et al., J. Virol. Method. (1994) 49:47-58; PCR, Essential Techniques (ed. J. F. Burke, J. Wiley & Sons, New York) (1996) pp. 61-63, 80-81; U.S. Pat. Nos. 5,723,591, 6,468,775, 6,277,607, 6,150,097, 6,174,670, 6,037,130, 6,399,320, 5,310,652, 6,300,073; U.S. Patent Appl. Nos. 2002/0119465A1; EP1132470A1; WO2000/71739A1; PCR Technology: Principles and Applications for DNA Amplification. Karl Drlica, John Wiley and Sons, 1997), direct cloning of PCR products (U.S. Pat. Nos. 5,827,657, 5,487,993), sequencing (U.S. Pat. Nos. 5,075,216, 4,795,699, 5,885,813, 4,994,372, 5,332,666, 5,498,523, 5,800,996, 5,821,058, 5,86,3727, 5,945,526, 6,258,568, 6,210,891, 6,274,320, 6,258,568; U.S. Patent Appl. Nos. 20020120126, 20020120127, 20020127552, 20030099972, 20030124594, and 20030207265 ; Sanger et al., 1977, Proc. Natl. Acad. Sci. USA, 74: 5463-5467; Sanger, 1981, Science, 214: 1205-1210; Ronaghi et al., 1998, Science 281:363, 365; Mitra et al., 2003, Analytical Biochemistry 320:55-65; Zhu et al., 2003, Science 301:836-8; Sambrook & Russell, Molecular Cloning: A Laboratory Manual 12.1-120 (3d Cold Spring Harbor Laboratory Press (ISBN: 0879695773)), mutagenesis, primer extension (Sambrook & Russell, Molecular Cloning: A Laboratory Manual 7.75-8.126, 13.1-105, A4.11-A4.29 (3d Cold Spring Harbor Laboratory Press (ISBN: 0879695773)).

The disclosure also provides kits comprising a package unit having a container comprising a chimeric polypeptide as disclosed herein. In some embodiments, a packaging unit can include a container comprising a polynucleotide having a sequence suitable for expressing a chimeric polypeptide. In some embodiments, a packaging unit can include a container comprising one or more reagents suitable for practicing one of the disclosed methods of using and/or making a chimeric polypeptide. Non-limiting of examples of reagents can be dNTPs, templates, vectors, primers, buffers, controls, host cells, host cell culture media, etc. In some embodiments, kits may include containers of reagents mixed together in suitable proportions for performing the methods described herein, including methods of making and using chimeric polymerases. In some embodiments, reagent containers can contain reagents in unit quantities that obviate measuring steps when performing the disclosed methods.

Aspects of the present disclosure may be further understood in light of the following examples, which should not be construed as limited the scope of the present disclosure in any way.

### 6. EXAMPLES

### Example 1

Chimeric Archeal B-Family Polymerases

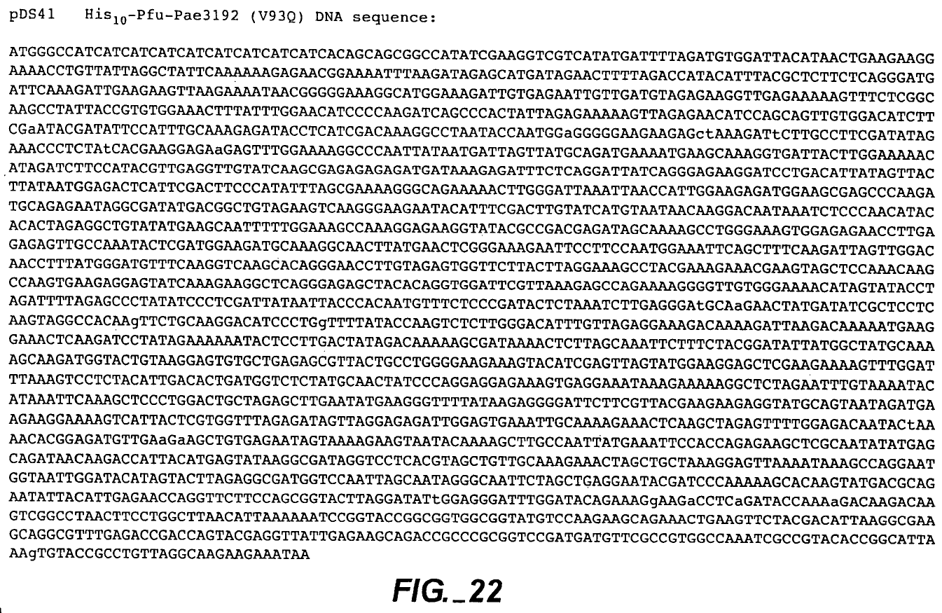

Two chimeric Pfu polymerases (Pfu-Pae3192; Pfu-Pae3192(V93Q) (FIG. 21-22) were produced by joining the sequence encoding Pfu polymerase in frame at its 3′ end with the nucleic acid sequence encoding non-specific double-stranded DNA binding protein, Pae3192. The chimeric polynucleotide was transformed into the Rosetta version of the BL21 (DE3) set of expression strains and recombinantly produced. To produce Pfu-Pae3192(V93Q), the encoding nucleic acid sequence was mutagenized by replacing the valine codon corresponding to position 93 of Pfu polymerase with a glutamine codon. The enzymatic activities of the chimeric polymerases were tested by a standard PCR of a 500 base pair sequence of λ genomic DNA in the presence of varying ratios of dTTP/dUTP (0%, 0.39%, 0.78%, 1.56%, 3.125%, 6.25%, 12.5%, 50% and 100%), PCR was performed in 50 μl V_{ƒ} containing 0.4 ng/μl λ DNA, 200 μM each dATP, dCTP, dGTP and the indicated ratios of dTTP/dUTP, 1× Phusion HF reaction buffer, 0.2 μM each

forward

(SEQ ID NO:77)

(L500F: 5′-AGCCAAGGCCAATATCTAAGTAAC-3′)
and
reverse

(SEQ ID NO:78)

(L500R: 5′-CGAAGCATTGGCCGTAAGTG-3′) primers.

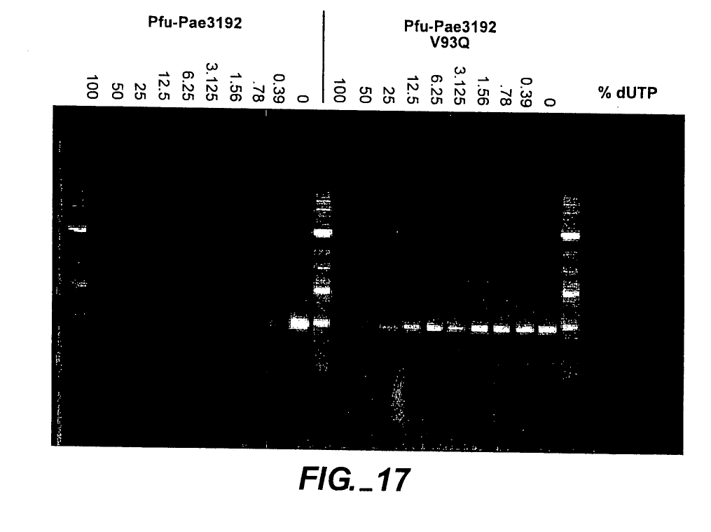

The reaction was cycled 25 times at 98° C. for 10 sec., 62° C. for 20 sec., and 72° C. for 20 sec. The results shown in FIG. 17 indicate that chimeric polymerase Pfu-Pae3192 was resistant to uracil up to about 0.39% dTTP/dUTP. Pfu-Pae3192(V93Q), which has descreased read-ahead function was substantially resistant to uracil at ratios of about 25-50% dTTP/dUTP.

The activity of chimeric fusions, Pfu-Pae3192 with and without the His-tag were compared. Preliminary results indicate that the non His-tagged version exhibited up to 50-fold less activity when compared to the His-tagged version.

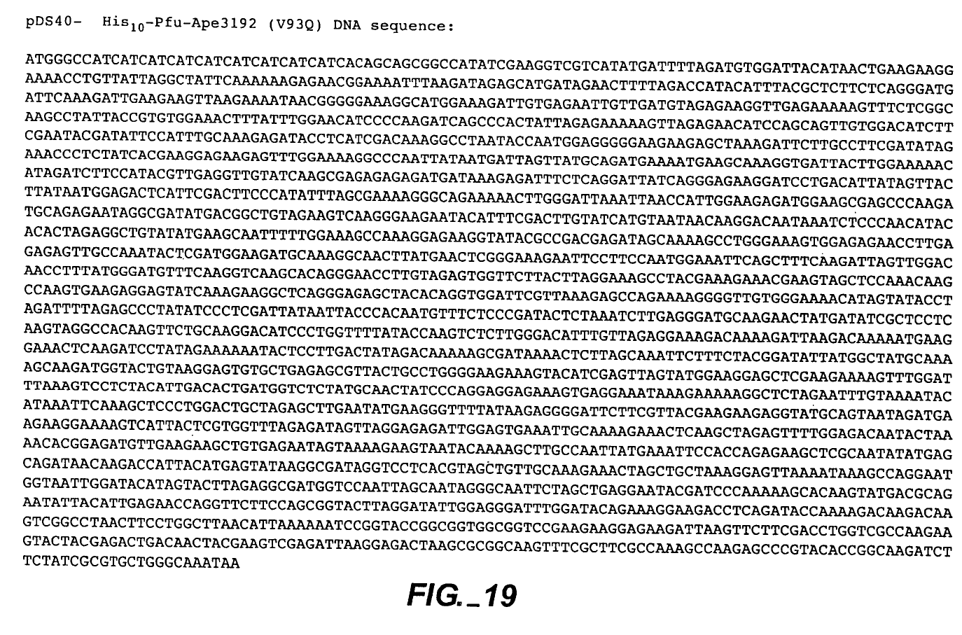

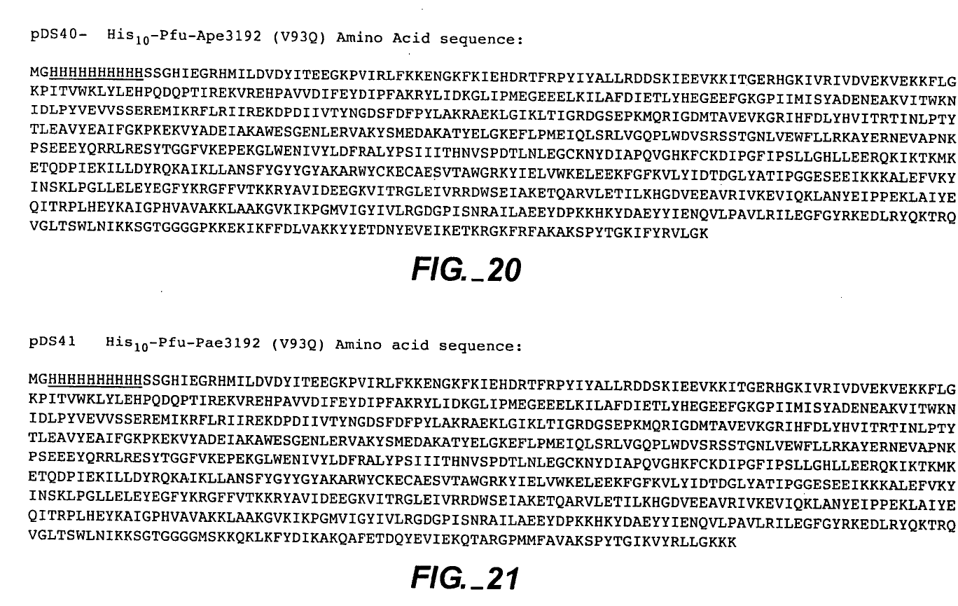

Chimeric Pfu polymerases (Pfu-Ape3192; Pfu-Ape3192(V93Q) (FIG. 19-20) are produced by joining the sequence encoding the Pfu polymerase in frame at its 3′ end with the nucleic acid sequence encoding non-specific DNA binding protein, Ape3192 similarly to the method described above for the Pfu-Pae3192 fusions. The Pfu-Ape3192 fusions with and without the histidine tags are tested for uracil resistance as described above.

### Example 2

Synthesis of a DUTPase Chimeric Polymerase

A thermostable dUTPase is assembled from synthetic oligonucleotides, cloned and fused in frame to either the N-terminus or C-terminus of Pfu polymerase. The Pfu polymerase is cloned into a T7-compatible expression systems. The dUTPase is assembled using the set of oligonucleotides shown in FIG. 18 using standard techniques.

The chimeric gene is transformed into the Rosetta version of the BL21(DE3) set of expression strains and recombinantly produced. The ability of the chimeric polymerase to produce PCR amplicons in the presence of varying amounts of dUTP is assessed as described in Example 1.

### Example 3

Synthesis of Chimeric B-Family Polymerases Lacking 3′→5′ Exonuclease Activity

The polynucleotides encoding the chimeric polymerases of Example 1 (FIGS. 19, 22) are mutated to produce a chimeric polymerase comprising D215A mutation which substantially reduce the 3′→5′ exonuclease activity. Alternatively, the oligonucleotides below are synthesized to incorporate phosphorothioate linkages between the last 3 bases at the 3′ end of each oligonucleotide. The ability of the chimeric polypeptide comprising the D215A mutation to progress past a dU residue in a DNA template is assessed using a primer extension assay as described by Fogg et al. Nature Struct Biol. 2002; 9:922-927, using the following oligonucleotides:

A:

(SEQ ID NO:79)

(VIC)-GGGGATCCTCTAGAGTCGACCTGC
B:

(SEQ ID NO:80)

(VIC)-GGAGACAAGCTTG(U/T)ATGCCTGCAGGTCGACTCTAGCGGCT
AAA.

While various specific embodiments have been illustrated and described, it will be appreciated that various changes can be made without departing from the spirit and scope of the invention(s).

1. A chimeric polypeptide comprising a polymerizing domain and a dUTPase domain.

2. The chimeric polypeptide of claim 1, wherein said polymerizing domain is positioned amino terminal to said dUTPase domain.

3. The chimeric polypeptide of claim 1, which further comprises a base analog detection domain.

4. The chimeric polypeptide of claim 3, which comprises a mutation that substantially inactivates said base analog detection domain.

5-12. (canceled)

13. The chimeric polypeptide of claim 1, which further comprises a 3′→5′ exonuclease domain.

14. The chimeric polypeptide of claim 13, which comprises one or more mutations that substantially inactivate said exonuclease domain.

15-22. (canceled)

23. The chimeric polypeptide of claim 1, which is thermostable.

24. The chimeric polypeptide of claim 1, wherein said polymerizing domain is a type B polymerizing domain.

25. The chimeric polypeptide of claim 24, wherein said type B polymerizing domain comprises an amino acid sequence that has at least about 95% identity with an archaebacterium polymerase.

26. The chimeric polypeptide of claim 1, wherein said dUTPase domain comprises an amino acid sequence has at least about 95% identity with an archaebacterium dUTPase.

27 and 28. (canceled)

29. A chimeric polypeptide comprising a type B polymerizing domain and a dUTPase domain, wherein said polymerizing domain is positioned amino terminal to said dUTPase domain and said chimeric polypeptide is thermostable.

30. The chimeric polypeptide of 29, which further comprises a non-specific DNA binding domain.

31-40. (canceled)

41. The chimeric polypeptide of claim 29, which further comprises a 3′→5′ exonuclease domain.

42. The chimeric polypeptide of claim 41, which comprises one or more mutations that substantially inactivate said exonuclease domain.

43-46. (canceled)

47. The chimeric polypeptide of claim 29, wherein said type B polymerizing domain comprises an amino acid sequence that has at least about 95% identity with an archaebacterium polymerase.

48. The chimeric polypeptide of claim 29, wherein said dUTPase domain comprises an amino acid sequence that has at least about 95% identity with an archaebacterium dUTPase.

49 and 50. (canceled)

51. A chimeric polypeptide comprising at least a type B polymerizing domain with reduced base analog detection activity and a non-specific nucleic acid binding domain that is at least about 95% identical to the amino acid sequence of Pae3192 or Ape3192.

52. The chimeric polypeptide of claim 51, which further comprises a dUTPase domain.

53. The chimeric polypeptide of claim 52, wherein said dUTPase domain is positioned carboxy terminal to said binding domain.

54. The chimeric polypeptide of claim 53, wherein said dUTPase domain has at least about 95% identity with an archaebacterium dUTPase.

55-87. (canceled)


SEQUENCE LISTING (97 sequences)

SEQ ID NO: 1 - protein, 50 aa, Pyrococcus abyssi
VQKKFLGRPIEVWKLYLEHPQDVPAIREKIREHPAVVDIFEYDIPFAKRY

SEQ ID NO: 2 - protein, 50 aa, Pyrococcus furiosus
VEKKFLGKPITVWKLYLEHPQDVPTIREKVREHPAVVDIFEYDIPFAKRY

SEQ ID NO: 3 - protein, 50 aa, Pyrococcus sp.
VRKKFLGRPIEVWRLYFEHPQDVPAIRDKIREHSAVIDIFEYDIPFAKRY

SEQ ID NO: 4 - protein, 50 aa, Pyrococcus glycovorans
VKKKFLGRPIEVWKLYFEHPQDVPAIRDKIREHPAVVDIFEYDIPFAKRY

SEQ ID NO: 5 - protein, 50 aa, Pyrococcus sp.
VSKKFLGRPIEVWKLYFEHPQDVPAIRDKIREHPAVIDIFEYDIPFAKRY

SEQ ID NO: 6 - protein, 50 aa, Thermococcus sp.
VQKKFLGRPIEVWKLYFNHPQDVPAIRDRIRAHPAVVDIYEYDIPFAKRY

SEQ ID NO: 7 - protein, 50 aa, Thermococcus fumicolans
VKKKFLGRPIEVWKLYFTHPQDVPAIRDKIREHPAVVDIYEYDIPFAKRY

SEQ ID NO: 8 - protein, 50 aa, Thermococcus gorgonarius
VKKKFLGRPIEVWKLYFTHPQDVPAIRDKIKEHPAVVDIYEYDIPFAKRY

SEQ ID NO: 9 - protein, 50 aa, Thermococcus hydrothermalis
VKKKFLGRPIEVWKLYFTHPQDVPAIRDEIRRHSAVVDIYEYDIPFAKRY

SEQ ID NO: 10 - protein, 50 aa, Thermococcus sp.
VKKKFLGRSVEVWVLYFTHPQDVPAIRDKIRKHPAVIDIYEYDIPFAKRY

SEQ ID NO: 11 - protein, 50 aa, Thermococcus kodakarensis
VQKKFLGRPVEVWKLYFTHPQDVPAIRDKIREHPAVIDIYEYDIPFAKRY

SEQ ID NO: 12 - protein, 50 aa, Thermococcus litoralis
VRKKFLGREVEVWKLIFEHPQDVPAMRGKIREHPAVVDIYEYDIPFAKRY

SEQ ID NO: 13 - protein, 50 aa, Thermococcus profundus
VKKKFLGRPIEVWKLYFTHPQDVPAIRDKIRKHPAVVDIYEYDIPFAKRY

SEQ ID NO: 14 - protein, 68 aa, Sulfolobus sulfataricus
MEISMATVKFKYKGEEKEVDISKIKKVWRVGKMISFTYDEGGGKTGRGAVSEKDAPKELLQMLEKQKK

SEQ ID NO: 15 - protein, 64 aa, Sulfolobus sulfataricus
MATVKFKYKGEEKQVDISKIKKVWRVGKMISFTYDEGGGKTGRGAVSEKDAPKELLQMLEKQKK

SEQ ID NO: 16 - protein, 64 aa, Sulfolobus sulfataricus; identical to SEQ ID NO: 15

SEQ ID NO: 17 - protein, 116 aa, Sulfolobus sulfataricus
MSIEISEKSFLLKRFLIVAYGLSEADVDAFIKIVSSETGKDVDAIAGELGISKSRASLILKKLADAGLVEKEKTSVSRGGRPKFLYRINKEELKKKLIKRSEETCKDLHTIISSFL

SEQ ID NO: 18 - protein, 100 aa, Sulfolobus sulfataricus
MEKMSSGTPTPSNVVLIGKKPVMNYVLAALTLLNQGVSEIVIKARGRAISKAVDTVEIVRNRFLPDKIEIKEIRVGSQVVTSQDGRQSRVSTIEIAIRKK

SEQ ID NO: 19 - protein, 89 aa, Sulfolobus sulfataricus
MTEKLNEIVVRKTKNVEDHVLDVIVLFNQGIDEVILKGTGREISKAVDVYNSLKDRLGDGVQLVNVQTGSEVRDRRRISYILLRLKRVY

SEQ ID NO: 20 - protein, 249 aa, Pyrococcus furiosus
MPFEIVFEGAKEFAQLIDTASKLIDEAAFKVTEDGISMRAMDPSRVVLIDLNLPSSIFSKYEVVEPETIGVNMDHLKKILKRGKAKDTLILKKGEENFLEITIQGTATRTFRVPLIDVEEMEVDLPELPFTAKVVVLGEVLKDAVKDASLVSDSIKFIARENEFIMKAEGETQEVEIKLTLEDEGLLDIEVQEETKSAYGVSYLSDMVKGLGKADEVTIKFGNEMPMQMEYYIRDEGRLTFLLAPRVEE

SEQ ID NO: 21 - protein, 57 aa, Pyrobaculum aerophilum
MSKKQKLKFYDIKAKQAFETDQYEVIEKQTARGPMMFAVAKSPYTGIKVYRLLGKKK

SEQ ID NO: 22 - protein, 57 aa, Pyrobaculum aerophilum; identical to SEQ ID NO: 21

SEQ ID NO: 23 - protein, 56 aa, Pyrobaculum aerophilum
MAKQKLKFYDIKAKQSFETDKYEVIEKETARGPMLFAVATSPYTGIKVYRLLGKKK

SEQ ID NO: 24 - protein, 55 aa, Aeropyrum pernix
MPKKEKIKFFDLVAKKYYETDNYEVEIKETKRGKFRFAKAKSPYTGKIFYRVLGK

SEQ ID NO: 25 - protein, 775 aa, Pyrococcus furiosus
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 26 - DNA, 2325 bp, Thermococcus kodakarensis
atgatcctcgacactgactacataaccgaggatggaaagcctgtcataagaattttcaagaaggaaaacggcgagtttaagattgagtacgaccggacttttgaaccctacttctacgccctcctgaaggacgattctgccattgaggaagtcaagaagataaccgccgagaggcacgggacggttgtaacggttaagcgggttgaaaaggttcagaagaagttcctcgggagaccagttgaggtctggaaactctactttactcatccgcaggacgtcccagcgataagggacaagatacgagagcatccagcagttattgacatctacgagtacgacatacccttcgccaagcgctacctcatagacaagggattagtgccaatggaaggcgacgaggagctgaaaatgctcgccttcgacattgaaactctctaccatgagggcgaggagttcgccgaggggccaatccttatgataagctacgccgacgaggaaggggccagggtgataacttggaagaacgtggatctcccctacgttgacgtcgtctcgacggagagggagatgataaagcgcttcctccgtgttgtgaaggagaaagacccggacgttctcataacctacaacggcgacaacttcgacttcgcctatctgaaaaagcgctgtgaaaagctcggaataaacttcgccctcggaagggatggaagcgagccgaagattcagaggatgggcgacaggtttgccgtcgaagtgaagggacggatacacttcgatctctatcctgtgataagacggacgataaacctgcccacatacacgcttgaggccgtttatgaagccgtcttcggtcagccgaaggagaaggtttacgctgaggaaataaccacagcctgggaaaccggcgagaaccttgagagagtcgcccgctactcgatggaagatgcgaaggtcacatacgagcttgggaaggagttccttccgatggaggcccagctttctcgcttaatcggccagtccctctgggacgtctcccgctccagcactggcaacctcgttgagtggttcctcctcaggaaggcctatgagaggaatgagctggccccgaacaagcccgatgaaaaggagctggccagaagacggcagagctatgaaggaggctatgtaaaagagcccgagagagggttgtgggagaacatagtgtacctagattttagatccctgtacccctcaatcatcatcacccacaacgtctcgccggatacgctcaacagagaaggatgcaaggaatatgacgttgccccacaggtcggccaccgcttctgcaaggacttcccaggatttatcccgagcctgcttggagacctcctagaggagaggcagaagataaagaagaagatgaaggccacgattgacccgatcgagaggaagctcctcgattacaggcagagggccatcaagatcctggcaaacagctactacggttactacggctatgcaagggcgcgctggtactgcaaggagtgtgcagagagcgtaacggcctggggaagggagtacataacgatgaccatcaaggagatagaggaaaagtacggctttaaggtaatctacagcgacaccgacggattttttgccacaatacctggagccgatgctgaaaccgtcaaaaagaaggctatggagttcctcaagtatatcaacgccaaacttccgggcgcgcttgagctcgagtacgagggcttctacaaacgcggcttcttcgtcacgaagaagaagtatgcggtgatagacgaggaaggcaagataacaacgcgcggacttgagattgtgaggcgtgactggagcgagatagcgaaagagacgcaggcgagggttcttgaagctttgctaaaggacggtgacgtcgagaaggccgtgaggatagtcaaagaagttaccgaaaagctgagcaagtacgaggttccgccggagaagctggtgatccacgagcagataacgagggatttaaaggactacaaggcaaccggtccccacgttgccgttgccaagaggttggccgcgagaggagtcaaaatacgccctggaacggtgataagctacatcgtgctcaagggctctgggaggataggcgacagggcgataccgttcgacgagttcgacccgacgaagcacaagtacgacgccgagtactacattgagaaccaggttctcccagccgttgagagaattctgagagccttcggttaccgcaaggaagacctgcgctaccagaagacgagacaggttggtttgagtgcttggctgaagccgaagggaacttga

SEQ ID NO: 27 - protein, 774 aa, Thermococcus litoralis
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 28 - protein, 775 aa, Pyrococcus sp.
MILDADYITEDGKPIIRIFKKENGEFKVEYDRNFRPYIYALLKDDSQIDEVRKITAERHGKIVRIIDAEKVRKKFLGRPIEVWRLYFEHPQDVPAIRDKIREHSAVIDIFEYDIPFAKRYLIDKGLIPMEGDEELKLLAFDIETLYHEGEEFAKGPIIMISYADEEEAKVITWKKIDLPYVEVVSSEREMIKRFLKVIREKDPDVIITYNGDSFDLPYLVKRAEKLGIKLPLGRDGSEPKMQRLGDMTAVEIKGRIHFDLYHVIRRTINLPTYTLEAVYEAIFGKPKEKVYAHEIAEAWETGKGLERVAKYSMEDAKVTYELGREFFPMEAQLSRLVGQPLWDVSRSSTGNLVEWYLLRKAYERNELAPNKPDEREYERRLRESYAGGYVKEPEKGLWEGLVSLDFRSLYPSIIITHNVSPDTLNREGCREYDVAPEVGHKFCKDFPGFIPSLLKRLLDERQEIKRKMKASKDPIEKKMLDYRQRAIKILANSYYGYYGYAKARWYCKECAESVTAWGREYIEFVRKELEEKFGFKVLYIDTDGLYATIPGAKPEEIKKKALEFVDYINAKLPGLLELEYEGFYVRGFFVTKKKYALIDEEGKIITRGLEIVRRDWSEIAKETQAKVLEAILKHGNVEEAVKIVKEVTEKLSKYEIPPEKLVIYEQITRPLHEYKAIGPHVAVAKRLAARGVKVRPGMVIGYIVLRGDGPISKRAILAEEFDLRKHKYDAEYYIENQVLPAVLRILEAFGYRKEDLRWQKTKQTGLTAWLNIKKK

SEQ ID NO: 29 - protein, 773 aa, Thermococcus gorgonarius
MILDTDYITEDGKPVIRIFKKENGEFKIDYDRNFEPYIYALLKDDSAIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRAIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 30 - protein, 781 aa, Archaeoglobus fulgidus
MERVEGWLIDADYETIGGKAVVRLWCKDDQGIFVAYDYNFDPYFYVIGVDEDILKNAATSTRREVIKLKSFEKAQLKTLGREVEGYIVYAHHPQHVPKLRDYLSQFGDVREADIPFAYRYLIDKDLACMDGIAIEGEKQGGVIRSYKIEKVERIPRMEFPELKMLVFDCEMLSSFGMPEPEKDPIIVISVKTNDDDEIILTGDERKIISDFVKLIKSYDPDIIVGYNQDAFDWPYLRKRAERWNIPLDVGRDGSNVVFRGGRPKITGRLNVDLYDIAMRISDIKIKKLENVAEFLGTKIEIADIEAKDIYRYWSRGEKEKVLNYARQDAINTYLIAKELLPMHYELSKMIRLPVDDVTRMGRGKQVDWLLLSEAKKIGEIAPNPPEHAESYEGAFVLEPERGLHENVACLDFASMYPSIMIAFNISPDTYGCRDDCYEAPEVGHKFRKSPDGFFKRILRMLIEKRRELKVELKNLSPESSEYKLLDIKQQTLKVLTNSFYGYMGWNLARWYCHPCAEATTAWGRHFIRTSAKIAESMGFKVLYGDTDSIFVTKAGMTKEDVDRLIDKLHEELPIQIEVDEYYSAIFFVEKKRYAGLTEDGRLVVKGLEVRRGDWCELAKKVQREVIEVILKEKNPEKALSLVKDVILRIKEGKVSLEEVVIYKGLTKKPSKYESMQAHVKAALKAREMGIIYPVSSKIGYVIVKGSGNIGDRAYPIDLIEDFDGENLRIKTKSGIEIKKLDKDYYIDNQIIPSVLRILERFGYTEASLKGSSQMSLDSFFS

SEQ ID NO: 31 - DNA, 60 bp, Artificial Sequence
  Description of Artificial Sequence Synthetic oligonucleotide
atgctgctgccggactggaaaatccgtaaagaaatcctgatcgaaccgttctctgaagaa

SEQ ID NO: 32 - DNA, 60 bp, Artificial Sequence
  Description of Artificial Sequence Synthetic oligonucleotide
tctctgcagccggctggttacgacctgcgtgttggtcgtgaagctttcgttaaaggtaaa

SEQ ID NO: 33 - DNA, 60 bp, Artificial Sequence
  Description of Artificial Sequence Synthetic oligonucleotide
ctgatcgacgttgaaaaagaaggtaaagttgttatcccgccgcgtgaatacgctctgatc

SEQ ID NO: 34 - DNA, 60 bp, Artificial Sequence
  Description of Artificial Sequence Synthetic oligonucleotide
ctgaccctggaacgtatcaaactgccggacgacgttatgggtgacatgaaaatccgttct

SEQ ID NO: 35 - DNA, 60 bp, Artificial Sequence
  Description of Artificial Sequence Synthetic oligonucleotide
tctctggctcgtgaaggtgttatcggttctttcgcttgggttgacccgggttgggacggt

SEQ ID NO: 36 - DNA, 60 bp, Artificial Sequence
  Description of Artificial Sequence Synthetic oligonucleotide
aacctgaccctgatgctgtacaacgcttctaacgaaccggttgaactgcgttacggtgaa

SEQ ID NO: 37 - DNA, 60 bp, Artificial Sequence
  Description of Artificial Sequence Synthetic oligonucleotide
cgtttcgttcagatcgctttcatccgtctggaaggtccggctcgtaacccgtaccgtggt

SEQ ID NO: 38 - DNA, 51 bp, Artificial Sequence
  Description of Artificial Sequence Synthetic oligonucleotide
aactaccagggttctacccgtctggctttctctaaacgtaaaaaactgtaa

SEQ ID NO: 39 - DNA, 20 bp, Artificial Sequence
  Description of Artificial Sequence Synthetic oligonucleotide
gctgcagagattcttcagag

SEQ ID NO: 40 - DNA, 20 bp, Artificial Sequence
  Description of Artificial Sequence Synthetic oligonucleotide
cgtcgatcagtttaccttta

SEQ ID NO: 41 - DNA, 20 bp, Artificial Sequence
  Description of Artificial Sequence Synthetic oligonucleotide
ccagggtcaggatcagagcg

SEQ ID NO: 42 - DNA, 20 bp, Artificial Sequence
  Description of Artificial Sequence Synthetic oligonucleotide
gagccagagaagaacggatt

SEQ ID NO: 43 - DNA, 20 bp, Artificial Sequence
  Description of Artificial Sequence Synthetic oligonucleotide
gggtcaggttaccgtcccaa

SEQ ID NO: 44 - DNA, 20 bp, Artificial Sequence
  Description of Artificial Sequence Synthetic oligonucleotide
gaacgaaacgttcaccgtaa

SEQ ID NO: 45 - DNA, 20 bp, Artificial Sequence
  Description of Artificial Sequence Synthetic oligonucleotide
cctggtagttaccacggtac

SEQ ID NO: 46 - protein, 41 aa, Pyrococcus abyssi
PKMQRMGDSLAVEIKGRIHFDLFPVIRRTINLPTYTLEAVY

SEQ ID NO: 47 - protein, 41 aa, Pyrococcus furiosus
PKMQRIGDMTAVEVKGRIHFDLYHVITRTINLPTYTLEAVY

SEQ ID NO: 48 - protein, 41 aa, Pyrococcus sp.
PKMQRLGDMTAVEIKGRIHFDLYHVIRRTINLPTYTLEAVY

SEQ ID NO: 49 - protein, 41 aa, Pyrococcus glycovorans; identical to SEQ ID NO: 48

SEQ ID NO: 50 - protein, 41 aa, Pyrococcus sp.
PKMQRLGESLAVEIKGRIHFDLFPVIRRTINLPTYTLRTVY

SEQ ID NO: 51 - protein, 41 aa, Thermococcus sp.
PKIQRMGDRFAVEVKGRIHFDLYPVIRRTINLPTYTLHAVY

SEQ ID NO: 52 - protein, 41 aa, Thermococcus fumicolans
PKIQRMGDRFAVEVKGRIHFDLYPVIRHTINLPTYTLEAVY

SEQ ID NO: 53 - protein, 41 aa, Thermococcus gorgonarius
PKIQRMGDRFAVEVKGRIHFDLYPVIRRTINLPTYTLEAVY

SEQ ID NO: 54 - protein, 41 aa, Thermococcus hydrothermalis; identical to SEQ ID NO: 53

SEQ ID NO: 55 - protein, 41 aa, Thermococcus sp.
PKIQRMGDRFAVEVKGRVHFDLYPVIRRTINLPTYTLEAVY

SEQ ID NO: 56 - protein, 41 aa, Thermococcus kodakarensis; identical to SEQ ID NO: 53

SEQ ID NO: 57 - protein, 41 aa, Thermococcus litoralis
PKIQRMGDSFAVEIKGRIHFDLFPVVRRTINLPTYTLEAVY

SEQ ID NO: 58 - protein, 41 aa, Thermococcus profundus; identical to SEQ ID NO: 55

SEQ ID NO: 59 - protein, 9 aa, Pyrococcus abyssi
LDFRSLYPS

SEQ ID NO: 60 - protein, 9 aa, Pyrococcus furiosus
LDFRALYPS

SEQ ID NO: 61 - protein, 9 aa, Pyrococcus sp.
LDFRSLYPS

SEQ ID NO: 62 - protein, 9 aa, Pyrococcus glycovorans
LDFRSLYPS

SEQ ID NO: 63 - protein, 9 aa, Pyrococcus sp.
LDFRSLYPS

SEQ ID NO: 64 - protein, 9 aa, Thermococcus sp.
LDFRSLYPS

SEQ ID NO: 65 - protein, 9 aa, Thermococcus fumicolans
LDFRSLYPS

SEQ ID NO: 66 - protein, 9 aa, Thermococcus gorgonarius
LDFRSLYPS

SEQ ID NO: 67 - protein, 9 aa, Thermococcus hydrothermalis
LDFMSLYPS

SEQ ID NO: 68 - protein, 9 aa, Thermococcus sp.
LDFRSLYPS

SEQ ID NO: 69 - protein, 9 aa, Thermococcus kodakarensis
LDFRSLYPS

SEQ ID NO: 70 - protein, 9 aa, Thermococcus litoralis
LDFRSLYPS

SEQ ID NO: 71 - protein, 9 aa, Thermococcus profundus
LDFRSLYPS

SEQ ID NO: 72 - DNA, 2571 bp, Artificial Sequence
  Description of Artificial Sequence Synthetic nucleotide sequence
atgggccatcatcatcatcatcatcatcatcatcacagcagcggccatatcgaaggtcgtcatatgattttagatgtggattacataactgaagaaggaaaacctgttattaggctattcaaaaaagagaacggaaaatttaagatagagcatgatagaacttttagaccatacatttacgctcttctcagggatgattcaaagattgaagaagttaagaaaataacgggggaaaggcatggaaagattgtgagaattgttgatgtagagaaggttgagaaaaagtttctcggcaagcctattaccgtgtggaaactttatttggaacatccccaagatcagcccactattagagaaaaagttagagaacatccagcagttgtggacatcttcgaatacgatattccatttgcaaagagatacctcatcgacaaaggcctaataccaatggagggggaagaagagctaaagattcttgccttcgatatagaaaccctctatcacgaaggagaagagtttggaaaaggcccaattataatgattagttatgcagatgaaaatgaagcaaaggtgattacttggaaaaacatagatcttccatacgttgaggttgtatcaagcgagagagagatgataaagagatttctcaggattatcagggagaaggatcctgacattatagttacttataatggagactcattcgacttcccatatttagcgaaaagggcagaaaaacttgggattaaattaaccattggaagagatggaagcgagcccaagatgcagagaataggcgatatgacggctgtagaagtcaagggaagaatacatttcgacttgtatcatgtaataacaaggacaataaatctcccaacatacacactagaggctgtatatgaagcaatttttggaaagccaaaggagaaggtatacgccgacgagatagcaaaagcctgggaaagtggagagaaccttgagagagttgccaaatactcgatggaagatgcaaaggcaacttatgaactcgggaaagaattccttccaatggaaattcagctttcaagattagttggacaacctttatgggatgtttcaaggtcaagcacagggaaccttgtagagtggttcttacttaggaaagcctacgaaagaaacgaagtagctccaaacaagccaagtgaagaggagtatcaaagaaggctcagggagagctacacaggtggattcgttaaagagccagaaaaggggttgtgggaaaacatagtatacctagattttagagccctatatccctcgattataattacccacaatgtttctcccgatactctaaatcttgagggatgcaagaactatgatatcgctcctcaagtaggccacaagttctgcaaggacatccctggttttataccaagtctcttgggacatttgttagaggaaagacaaaagattaagacaaaaatgaaggaaactcaagatcctatagaaaaaatactccttgactatagacaaaaagcgataaaactcttagcaaattctttctacggatattatggctatgcaaaagcaagatggtactgtaaggagtgtgctgagagcgttactgcctggggaagaaagtacatcgagttagtatggaaggagctcgaagaaaagtttggatttaaagtcctctacattgacactgatggtctctatgcaactatcccaggaggagaaagtgaggaaataaagaaaaaggctctagaatttgtaaaatacataaattcaaagctccctggactgctagagcttgaatatgaagggttttataagaggggattcttcgttacgaagaagaggtatgcagtaatagatgaagaaggaaaagtcattactcgtggtttagagatagttaggagagattggagtgaaattgcaaaagaaactcaagctagagttttggagacaatactaaaacacggagatgttgaagaagctgtgagaatagtaaaagaagtaatacaaaagcttgccaattatgaaattccaccagagaagctcgcaatatatgagcagataacaagaccattacatgagtataaggcgataggtcctcacgtagctgttgcaaagaaactagctgctaaaggagttaaaataaagccaggaatggtaattggatacatagtacttagaggcgatggtccaattagcaatagggcaattctagctgaggaatacgatcccaaaaagcacaagtatgacgcagaatattacattgagaaccaggttcttccagcggtacttaggatattggagggatttggatacagaaaggaagacctcagataccaaaagacaagacaagtcggcctaacttcctggcttaacattaaaaaatccggtaccggcggtggcggtccgaagaaggagaagattaagttcttcgacctggtcgccaagaagtactacgagactgacaactacgaagtcgagattaaggagactaagcgcggcaagtttcgcttcgccaaagccaagagcccgtacaccggcaagatcttctatcgcgtgctgggcaaataa

SEQ ID NO: 73 - protein, 856 aa, Artificial Sequence
  Description of Artificial Sequence Synthetic protein sequence
MGHHHHHHHHHHSSGHIEGRHMILDVDYITEEGKPVIRLFKKENGKFKIEHDRTFRPYIYALLRDDSKIEEVKKITGERHGKIVRIVDVEKVEKKFLGKPITVWKLYLEHPQDQPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKSGTGGGGPKKEKIKFFDLVAKKYYETDNYEVEIKETKRGKFRFAKAKSPYTGKIFYRVLGK

SEQ ID NO: 74 - protein, 859 aa, Artificial Sequence
  Description of Artificial Sequence Synthetic protein sequence
MGHHHHHHHHHHSSGHIEGRHMILDVDYITEEGKPVIRLFKKENGKFKIEHDRTFRPYIYALLRDDSKIEEVKKITGERHGKIVRIVDVEKVEKKFLGKPITVWKLYLEHPQDQPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKSGTGGGGMSKKQKLKFYDIKAKQAFETDQYEVIEKQTARGPMMFAVAKSPYTGIKVYRLLGKKK

SEQ ID NO: 75 - DNA, 2580 bp, Artificial Sequence
  Description of Artificial Sequence Synthetic nucleotide sequence
atgggccatcatcatcatcatcatcatcatcatcacagcagcggccatatcgaaggtcgtcatatgattttagatgtggattacataactgaagaaggaaaacctgttattaggctattcaaaaaagagaacggaaaatttaagatagagcatgatagaacttttagaccatacatttacgctcttctcagggatgattcaaagattgaagaagttaagaaaataacgggggaaaggcatggaaagattgtgagaattgttgatgtagagaaggttgagaaaaagtttctcggcaagcctattaccgtgtggaaactttatttggaacatccccaagatcagcccactattagagaaaaagttagagaacatccagcagttgtggacatcttcgaatacgatattccatttgcaaagagatacctcatcgacaaaggcctaataccaatggagggggaagaagagctaaagattcttgccttcgatatagaaaccctctatcacgaaggagaagagtttggaaaaggcccaattataatgattagttatgcagatgaaaatgaagcaaaggtgattacttggaaaaacatagatcttccatacgttgaggttgtatcaagcgagagagagatgataaagagatttctcaggattatcagggagaaggatcctgacattatagttacttataatggagactcattcgacttcccatatttagcgaaaagggcagaaaaacttgggattaaattaaccattggaagagatggaagcgagcccaagatgcagagaataggcgatatgacggctgtagaagtcaagggaagaatacatttcgacttgtatcatgtaataacaaggacaataaatctcccaacatacacactagaggctgtatatgaagcaatttttggaaagccaaaggagaaggtatacgccgacgagatagcaaaagcctgggaaagtggagagaaccttgagagagttgccaaatactcgatggaagatgcaaaggcaacttatgaactcgggaaagaattccttccaatggaaattcagctttcaagattagttggacaacctttatgggatgtttcaaggtcaagcacagggaaccttgtagagtggttcttacttaggaaagcctacgaaagaaacgaagtagctccaaacaagccaagtgaagaggagtatcaaagaaggctcagggagagctacacaggtggattcgttaaagagccagaaaaggggttgtgggaaaacatagtatacctagattttagagccctatatccctcgattataattacccacaatgtttctcccgatactctaaatcttgagggatgcaagaactatgatatcgctcctcaagtaggccacaagttctgcaaggacatccctggttttataccaagtctcttgggacatttgttagaggaaagacaaaagattaagacaaaaatgaaggaaactcaagatcctatagaaaaaatactccttgactatagacaaaaagcgataaaactcttagcaaattctttctacggatattatggctatgcaaaagcaagatggtactgtaaggagtgtgctgagagcgttactgcctggggaagaaagtacatcgagttagtatggaaggagctcgaagaaaagtttggatttaaagtcctctacattgacactgatggtctctatgcaactatcccaggaggagaaagtgaggaaataaagaaaaaggctctagaatttgtaaaatacataaattcaaagctccctggactgctagagcttgaatatgaagggttttataagaggggattcttcgttacgaagaagaggtatgcagtaatagatgaagaaggaaaagtcattactcgtggtttagagatagttaggagagattggagtgaaattgcaaaagaaactcaagctagagttttggagacaatactaaaacacggagatgttgaagaagctgtgagaatagtaaaagaagtaatacaaaagcttgccaattatgaaattccaccagagaagctcgcaatatatgagcagataacaagaccattacatgagtataaggcgataggtcctcacgtagctgttgcaaagaaactagctgctaaaggagttaaaataaagccaggaatggtaattggatacatagtacttagaggcgatggtccaattagcaatagggcaattctagctgaggaatacgatcccaaaaagcacaagtatgacgcagaatattacattgagaaccaggttcttccagcggtacttaggatattggagggatttggatacagaaaggaagacctcagataccaaaagacaagacaagtcggcctaacttcctggcttaacattaaaaaatccggtaccggcggtggcggtatgtccaagaagcagaaactgaagttctacgacattaaggcgaagcaggcgtttgagaccgaccagtacgaggttattgagaagcagaccgcccgcggtccgatgatgttcgccgtggccaaatcgccgtacaccggcattaaagtgtaccgcctgttaggcaagaagaaataa

SEQ ID NO: 76 - protein, 8 aa, Artificial Sequence
  Description of Artificial Sequence Synthetic peptide
DYKDDDDK

SEQ ID NO: 77 - DNA, 24 bp, Artificial Sequence
  Description of Artificial Sequence Synthetic primer
agccaaggccaatatctaagtaac

SEQ ID NO: 78 - DNA, 20 bp, Artificial Sequence
  Description of Artificial Sequence Synthetic primer
cgaagcattggccgtaagtg

SEQ ID NO: 79 - DNA, 24 bp, Artificial Sequence
  Description of Artificial Sequence Synthetic oligonucleotide
ggggatcctctagagtcgacctgc

SEQ ID NO: 80 - DNA, 43 bp, Artificial Sequence
  Description of Combined DNA/RNA Molecule Synthetic oligonucleotide
  Description of Artificial Sequence Synthetic oligonucleotide
  modified_base (14): u or t
ggagacaagcttgnatgcctgcaggtcgactctagcggctaaa

SEQ ID NO: 81 - protein, 4 aa, Artificial Sequence
  Description of Artificial Sequence Synthetic peptide
DIET

SEQ ID NO: 82 - protein, 4 aa, Artificial Sequence
  Description of Artificial Sequence Synthetic peptide
DIDT

SEQ ID NO: 83 - protein, 4 aa, Artificial Sequence
  Description of Artificial Sequence Synthetic peptide
AIAT

SEQ ID NO: 84 - protein, 13 aa, Pyrococcus abyssi
LLDYRQRAIKILA

SEQ ID NO: 85 - protein, 13 aa, Pyrococcus furiosus
LLDYRQKAIKLLA

SEQ ID NO: 86 - protein, 13 aa, Pyrococcus sp.
MLDYRQRAIKILA

SEQ ID NO: 87 - protein, 13 aa, Pyrococcus glycovorans
MLDYRQRAIKILA

SEQ ID NO: 88 - protein, 13 aa, Pyrococcus sp.
LLDFRQRAIKILA

SEQ ID NO: 89 - protein, 13 aa, Thermococcus sp.
LLDYRQRAIKILA

SEQ ID NO: 90 - protein, 13 aa, Thermococcus fumicolans
LLDYRQRAIKILA

SEQ ID NO: 91 - protein, 13 aa, Thermococcus gorgonarius
LLDYRQRAIKILA

SEQ ID NO: 92 - protein, 13 aa, Thermococcus hydrothermalis
LLDYRQKAIKILA

SEQ ID NO: 93 - protein, 13 aa, Thermococcus sp.
LLDYRQRAIKILA

SEQ ID NO: 94 - protein, 13 aa, Thermococcus kodakarensis
LLDYRQRAIKILA

SEQ ID NO: 95 - protein, 13 aa, Thermococcus litoralis
MLDYRQRAIKLLA

SEQ ID NO: 96 - protein, 13 aa, Thermococcus profundus
LLDYRQRAIKILA

SEQ ID NO: 97 - protein, 10 aa, Artificial Sequence
  Description of Artificial Sequence Synthetic peptide
HHHHHHHHHH